(19) United States
(12) Patent Application Publication
(10) Pub. No.: US 2024/0268664 A1
(43) Pub. Date: Aug. 15, 2024

(54) RETINAL IMAGING DEVICE FOR ASSESSING OCULAR TISSUE

(71) Applicants: Bingda LI, Pittsburgh, PA (US); Devansh Sureshkumar PARIKH, Vadodara (IN); Mukunthan THARMAKULASINGAM, London (GB)

(72) Inventors: Bingda LI, Pittsburgh, PA (US); Devansh Sureshkumar PARIKH, Vadodara (IN); Mukunthan THARMAKULASINGAM, London (GB)

(21) Appl. No.: 18/439,426

(22) Filed: Feb. 12, 2024

Related U.S. Application Data
(60) Provisional application No. 63/484,260, filed on Feb. 10, 2023.

(51) Int. Cl. A61B 3/12; A61B 3/00; A61B 3/14; G06T 7/00

(52) U.S. Cl. CPC A61B 3/12; A61B 3/0008; A61B 3/14; G06T 7/0012; G06T 2207/20081; G06T 2207/30041

(57) ABSTRACT

A system for processing a retinal image is disclosed. The system comprises a retinal imaging adapter, an imaging device, and a processor. The retinal imaging adapter and the imaging device are optically aligned with one another and configured to be optically aligned with an eye 10 of a patient. The imaging device is in electrical communication with the processor. The processor is configured to receive an image of a retina of a patient, identify one or more features of the retina using a machine learning algorithm based on the image, and determine a condition of the patient based on the one or more identified features. In some embodiments, the condition of the patient may relate to the health of the patient, a disease state of the patient (e.g., diabetic retinopathy (DR)), and/or a disease stage of the patient (e.g., a stage of DR).

### CROSS-REFERENCE TO RELATED APPLICATIONS

This application claims the benefit of priority to U.S. Provisional Application No. 63/484,260 entitled “Retinal Imaging Device for Assessing Ocular Tissue,” filed Feb. 10, 2023, which is incorporated herein by reference in its entirety.

### TECHNICAL FIELD

The present disclosure relates generally to methods, systems, and devices related to identifying and/or assessing ocular conditions. More particularly, the present disclosure relates to a system comprising a mobile device-compatible ophthalmoscope and accompanying software for assessing ocular conditions via a deep learning model. The disclosed techniques may be applied to, for example, diagnosing or assessing retinal conditions, e.g., diabetic retinopathy.

### BACKGROUND

In ophthalmology, the retina of the human eye is an important feature for assessment and/or diagnosis of a variety of conditions. For example, diabetic retinopathy (DR) or diabetic eye disease (DED) is a medical condition associated with diabetes mellitus that can damage the retina and is a leading cause of visual impairment worldwide among working-aged adults. Early detection of diabetic retinopathy is possible based on non-invasive images of the retina and timely treatment can prevent larger complications including blindness.

Moreover, while a vast majority of the human circulatory system require invasive techniques for direct visualization and/or assessment, the retina is in fact a part of the human circulatory system and is capable of being visualized and/or photographed in a non-invasive manner. As such, non-invasive imaging of the retina, e.g., the retinal fundus, may be a valuable tool that enables identification, characterization, and/or documentation of retinal conditions as well as broader systemic diseases and related microvascular complications through digital analysis. For example, physicians can utilize retinal images as a means for non-invasive in vivo assessment of retinal integrity, blood vessels, and the optic nerve head, which may facilitate detection of serious conditions such as malignant hypertension, elevated intracranial pressure, metastatic cancer, and/or more common eye conditions such as diabetic retinopathy or macular degeneration.

Currently, some methods for achieving an adequate image of the retinal fundus are established in the medical field. For example, an image of the retina may be obtained by a medical professional using an ophthalmoscope. However, in practice, several barriers exist that often prevent medical professionals from obtaining adequate images, and recent studies and research indeed support the finding that direct ophthalmoscopy is underutilized in the medical field, especially by professionals other than ophthalmologists.

A primary barrier may be the availability of ophthalmoscopes at the site of care. Ophthalmoscopes are specialized instruments that are not commonly available in practices outside of ophthalmology. Further, basic practice instruments are unable to reproduce the high image quality and complex characteristics elucidated by an ophthalmoscope. At the same time, recent findings have also shown that constant provision to an ophthalmoscope nonetheless failed to stimulate a significant increase in usage. This finding suggests that additional barriers to undertaking ophthalmoscopy may persist, e.g., time constraints; difficulty in discerning useful information from images due to image quality, poor training, or other factors; inadequate visibility, particularly with older patients having smaller pupils in well-illuminated environments; lack of training; lack of knowledge of the importance of ophthalmoscopy; and/or lack of confidence using an ophthalmoscope.

Another method of obtaining an image of the retinal fundus is known as fundus photography. Fundus photography involves photographing the rear of the eye using specialized fundus cameras consisting of an intricate microscope attached to a flash enabled camera, which enables clear visualization of the central and peripheral retina, optic disc, and macula. Generally, fundus photography is more widely accepted as opposed to direct ophthalmoscopy, especially amongst non-medical professionals, likely due to the technical simplicity, speed, and standardization involved in training on a fundus camera and obtaining images therewith. As a result, medical technicians and assistants are often more comfortable and capable of accurately identifying ocular fundus features using photographs from a fundus camera. Considering the shortages of medical and nursing staff in the current world climate as well as the increasing demand and prevalence of telehealth services, it would be advantageous for medical assistants to be able to obtain fundus photos for remote review and interpretation by an ophthalmic specialist. Furthermore, this capability would improve the availability of ophthalmic services in remote areas and low- and middle-income countries. Until now, a lack of resources has largely prevented diabetic retinopathy screening implementation in such areas despite approximately 80% of the affected population residing in such areas.

Accordingly, it would be advantageous to have a simple, portable fundus imaging system with sufficient image quality for assessment and diagnosis of ocular and systemic conditions, e.g., diabetic retinopathy. It would be further advantageous for such a system to simplify and/or automate imaging collection and diagnosis using the obtained fundus images.

### SUMMARY

An apparatus for capturing an image of an eye of a patient is provided. The apparatus comprises: a lens assembly comprising a first end, a second end, and a central optical axis extending between the first end and the second end, wherein the first end is configured to be aligned with a camera lens of a smart device, and wherein the second end is configured to interface with a surface adjacent the eye of the patient; and a mounting assembly configured to couple the smart device to the lens assembly to form an imaging device, wherein the mounting assembly is configured to adjust a position of the smart device with respect to the lens assembly, wherein the lens assembly is configured to transmit light from the eye of the patient to the camera lens when the smart device is coupled to the lens assembly such that the second end is aligned with the camera lens.

According to some embodiments, the smart device is one of a smartphone and a tablet.

According to some embodiments, the imaging device formed by the apparatus and the smart device is a digital fundus camera.

According to some embodiments, an optical axis of the camera lens coincides with the central optical axis when the smart device is coupled to the lens assembly.

According to some embodiments, the lens assembly comprises one or more lenses and one or more reflectors. According to additional embodiments, the lens assembly further comprises a light channel having a first end and a second end, wherein the first end of the light channel is configured to receive illuminating light from a flash unit of the smart device when the smart device is coupled to the lens assembly, and wherein the second end of the fiber optic tube is configured to emit the illuminating light to the eye of the patient. According to further embodiments, the light channel comprises a fiber optic tube. According to further embodiments, the apparatus further comprises a beam splitter configured to: deflect the illuminating light from the light channel to the eye of the patient; and transmit the light from the eye of the patient to the camera lens. According to additional embodiments, the apparatus further comprises one or more condensing lenses.

A system for processing an image of an eye of a patient is also provided. The system comprises: an imaging device comprising a camera lens; an imaging adapter comprising a lens assembly in optical communication with the camera lens, the lens assembly having a first end, a second end, and a central optical axis extending between the first end and the second end, wherein the first end is configured to be aligned with the camera lens, and wherein the second end is configured to interface with a surface adjacent the eye of the patient; a processor in electrical communication with the imaging device; and a non-transitory, computer-readable medium storing instructions that, when executed, cause the processor to: receive an image of the eye of the patient from the imaging device, identify one or more features of a retina of the eye using a machine learning algorithm based on the image, and determine a condition of the patient based on the one or more identified features.

According to some embodiments, the system further comprises a database in electrical communication with the processor, the database comprising ophthalmological data, wherein the machine learning algorithm identifies one or more features of the retina based further on the ophthalmological data. According to additional embodiments, the machine learning algorithm determines the condition of the patient based further on the ophthalmological data.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. Various aspects of at least one example are discussed below with reference to the accompanying drawings, which are not intended to be drawn to scale. In the drawings:

FIG. 1 depicts a frontal view of the retinal imaging adapter in accordance with an embodiment.

FIG. 2A depicts a first partial view of the retinal imaging adapter assembled with a smartphone to form a retinal imaging device, i.e., a digital fundus camera, in accordance with an embodiment.

FIG. 2B depicts a second view of the retinal imaging adapter assembled with a smartphone to form the retinal imaging device in accordance with an embodiment.

FIG. 3 depicts a partial view of a support and a mounting assembly of the retinal imaging adapter in accordance with an embodiment.

FIG. 4 depicts a cross-sectional view of the retinal imaging adapter in accordance with an embodiment.

FIG. 5 depicts a cross-sectional detailed view of a lens assembly of the retinal imaging adapter as part of an assembled imaging device in accordance with an embodiment.

FIG. 6 depicts a block diagram of an exemplary system for processing a retinal image (e.g., a fundus image) in accordance with an embodiment.

FIG. 7 depicts a flow diagram of an illustrative method for processing a retinal image in accordance with an embodiment.

FIG. 8 depicts a network within which a system for processing a retinal image may operate in accordance with an embodiment.

FIG. 9 depicts an exemplary retinal image captured using the devices, systems, and methods described herein.

FIG. 10 illustrates a block diagram of an exemplary data processing system in which embodiments are implemented.

FIGS. 11A-11B depict multiple views of an exemplary smartphone-compatible retinal imaging adapter in accordance with an alternate embodiment.

FIG. 12A depicts a first view of an exemplary imaging device 200 formed by a smartphone and the retinal imaging adapter of FIGS. 11A-11B in accordance with an alternate embodiment.

FIG. 12B depicts a partial cross-sectional view of an exemplary imaging device 200 formed by a smartphone and the retinal imaging adapter of FIGS. 11A-11B in accordance with an alternate embodiment.

FIG. 13A depicts exemplary lens assembly of the retinal imaging adapter, e.g., the retinal imaging adapter of FIGS. 11A-11B, in accordance with an alternate embodiment.

FIG. 13B depicts exemplary mounting assembly of the retinal imaging adapter, e.g., the retinal imaging adapter of FIGS. 11A-11B, in accordance with an alternate embodiment.

FIG. 14 depicts a partial cross-sectional view of an exemplary lens assembly of the retinal imaging adapter, e.g., the retinal imaging adapter of FIGS. 11A-11B, in accordance with an alternate embodiment.

FIG. 15 depicts a cross-sectional detailed view of a lens assembly of the retinal imaging adapter as part of an assembled imaging device in accordance with an embodiment.

FIG. 16 depicts a LIME-based explanation framework in accordance with an embodiment.

FIGS. 17A-17B depict exemplary LIME-generated images generated through a LIME-based explanation framework in accordance with some embodiments.

FIG. 18 depicts exemplary pre-processed images of a retina in accordance with some embodiments.

### DETAILED DESCRIPTION

This disclosure is not limited to the particular systems, devices and methods described, as these may vary. The terminology used in the description is for the purpose of describing the particular versions or embodiments only, and is not intended to limit the scope. Such aspects of the disclosure be embodied in many different forms; rather, these embodiments are provided so that this disclosure will be thorough and complete, and will fully convey its scope to those skilled in the art.

As used in this document, the singular forms “a,” “an,” and “the” include plural references unless the context clearly dictates otherwise. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

As will be understood by one skilled in the art, for any and all purposes, such as in terms of providing a written description, all ranges disclosed herein are intended as encompassing each intervening value between the upper and lower limit of that range and any other stated or intervening value in that stated range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells as well as the range of values greater than or equal to 1 cell and less than or equal to 3 cells. Similarly, a group having 1-5 cells refers to groups having 1, 2, 3, 4, or 5 cells, as well as the range of values greater than or equal to 1 cell and less than or equal to 5 cells, and so forth.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as “about 49, about 50, about 55”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). Further, the transitional term “comprising,” which is synonymous with “including,” “containing,” or “characterized by,” is inclusive or open-ended and does not exclude additional, unrecited elements or method steps. While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “patient” and “subject” are interchangeable and refer to any living organism which contains ocular and/or retinal tissue. As such, the terms “patient” and “subject” may include, but are not limited to, any non-human mammal, primate or human. A subject can be a mammal such as a primate, for example, a human. The term “subject” includes domesticated animals (e.g., cats, dogs, etc.); livestock (e.g., cattle, horses, swine, sheep, goats, etc.), and laboratory animals (e.g., mice, rabbits, rats, gerbils, guinea pigs, possums, etc.). A patient or subject may be an adult, child or infant.

The term “tissue” refers to any aggregation of similarly specialized cells which are united in the performance of a particular function.

The term “disorder” is used in this disclosure to mean, and is used interchangeably with, the terms “disease,” “condition,” or “illness,” unless otherwise indicated.

The term “real-time” is used to refer to calculations or operations performed on-the-fly as events occur or input is received by the operable system. However, the use of the term “real-time” is not intended to preclude operations that cause some latency between input and response, so long as the latency is an unintended consequence induced by the performance characteristics of the machine.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

Throughout this disclosure, various patents, patent applications and publications are referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

As discussed herein, it would be advantageous to have a simple, portable fundus imaging device providing sufficient image quality for assessment and diagnosis of ocular and systemic conditions. Ideally, the fundus imaging device would be compatible with widely available electronics, e.g., a smartphone or other portable electronic device (PED), in order to simplify the device requirements and reduce the cost thereof. In some embodiments, a fundus imaging system for assessing one or more health conditions may be formed using the fundus imaging device, a smartphone or other PED, and/or screening software configured for assessing images captured using the fundus imaging device.

### Smartphone-Compatible Retinal Imaging Adapter

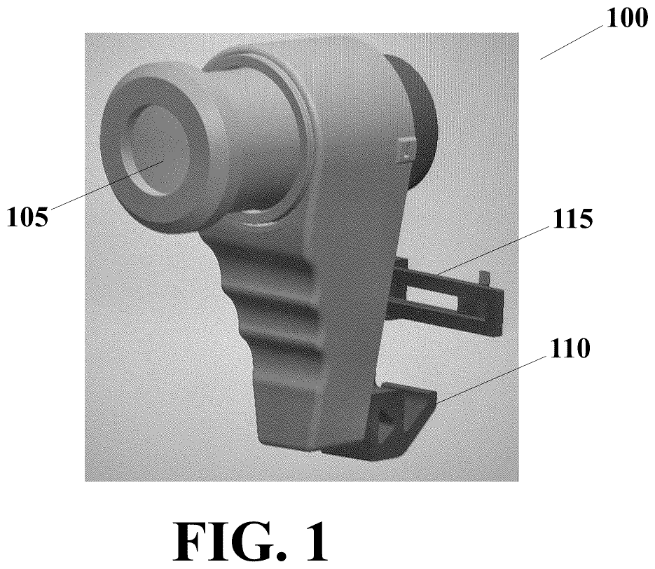

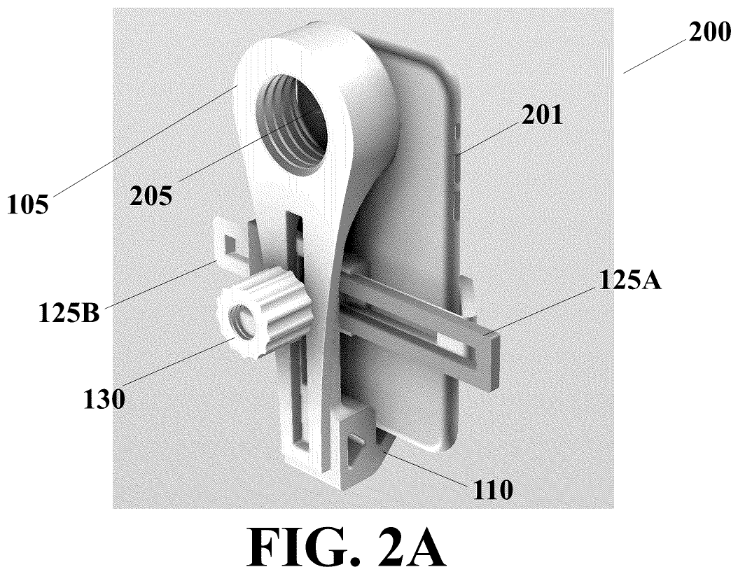

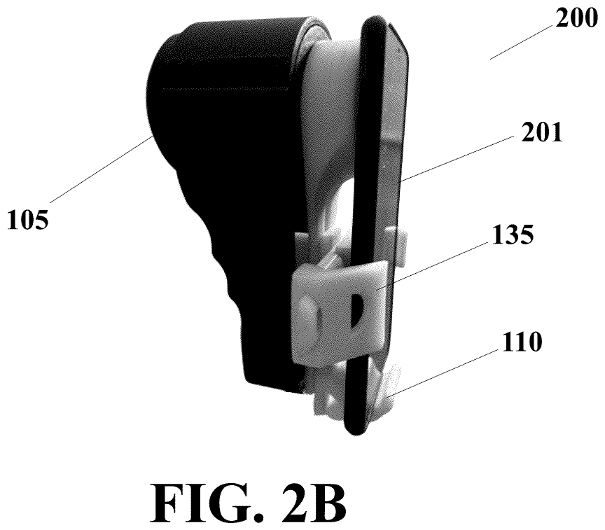

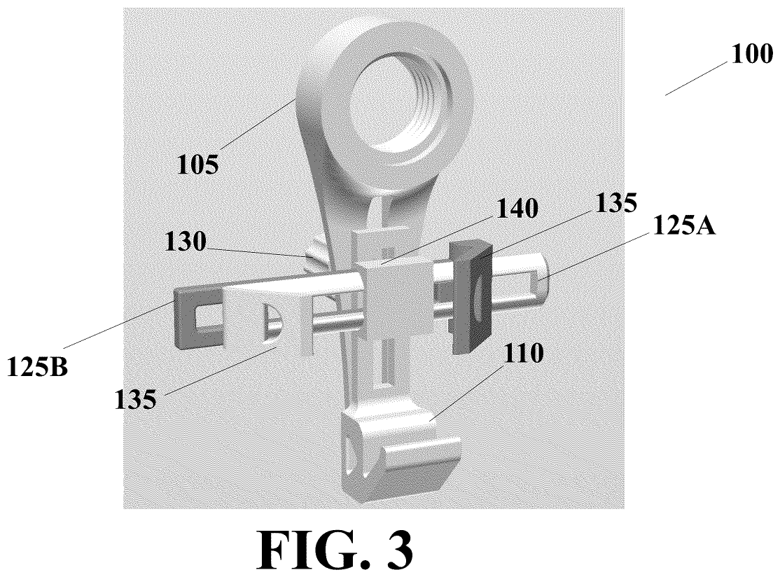

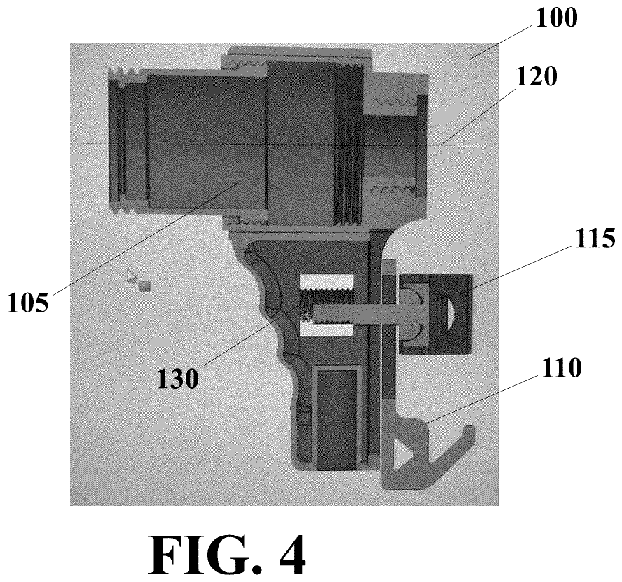

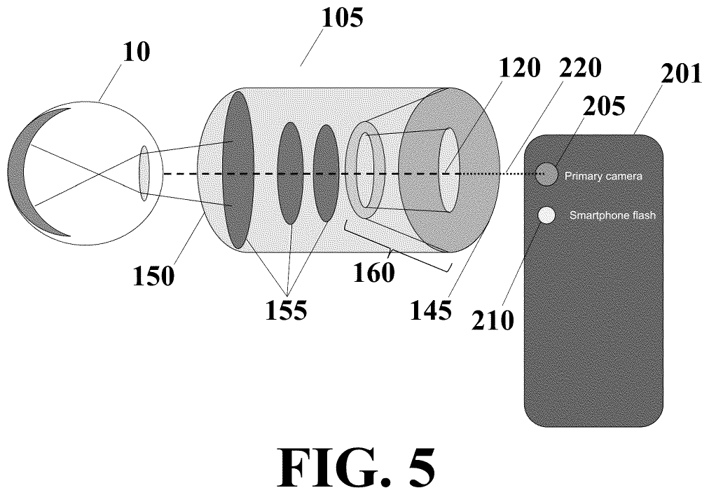

Turning now to FIGS. 1-5, an exemplary smartphone-compatible retinal imaging adapter is depicted in accordance with an embodiment. FIG. 1 depicts a frontal view of the retinal imaging adapter 100 in accordance with an embodiment. FIG. 2A depicts a first partial view of the retinal imaging adapter 100 assembled with a smartphone 201 to form a retinal imaging device 200, i.e., a digital fundus camera 200, in accordance with an embodiment. FIG. 2B depicts a second view of the retinal imaging adapter 100 assembled with a smartphone to form the retinal imaging device 200 in accordance with an embodiment. FIG. 3 depicts a partial view of a support and a mounting assembly of the retinal imaging adapter 100 in accordance with an embodiment. FIG. 4 depicts a cross-sectional view of the retinal imaging adapter 100 in accordance with an embodiment. FIG. 5 depicts a cross-sectional detailed view of a lens assembly of the retinal imaging adapter in accordance with an embodiment. Similar features within FIGS. 1-5 are identified with common reference numbers.

As shown in FIG. 1, the retinal imaging adapter 100 may be configured to be mounted over a camera lens of a smartphone. The adapter 100 comprises a lens assembly 105, a support 110, and a mounting assembly 115. It should be understood that the retinal imaging adapter 100, standing alone, may comprise an ophthalmoscope and may be interchangeably referred to as an ophthalmoscope 100 throughout this disclosure. As further described herein, when a smartphone 201 is assembled with the ophthalmoscope 100, the assembly comprising the retinal imaging device 200 may form a digital fundus camera and thus the retinal imaging device 200 may be interchangeably referred to as a digital fundus camera 200 throughout this disclosure.

The lens assembly 105 may be configured to be aligned with a camera lens of a smartphone. For example, the lens assembly 105 and the camera lens may be arranged in series. In some embodiments, as shown in FIG. 4, the lens assembly comprises a central optical axis 120 along which light may be passed to collect an image in the manner of a conventional camera lens. Accordingly, as shown in FIGS. 2A-2B and 5, a camera lens 205 of a smartphone 201 may be aligned with an end of the lens assembly 105 such that an optical axis 220 of the camera lens 205 is coincident or co-extensive with the central optical axis 120. For example, FIG. 5 depicts the smartphone 201 being aligned with the lens assembly 105 such that optical axis 220 (depicted as a dotted line) of the camera lens 205 is co-extensive with the central optical axis 120 (depicted as a dashed line) of the lens assembly 105. As such, light reaching the camera lens 205 may be modified by diffraction through the lens assembly 105 prior to reaching the camera lens 205 and thus images obtained by the smartphone 201 may be modified by the lens assembly 105.

In some embodiments, the adapter 100 is configured to mount to the smartphone 201 in an adjustable manner. As smartphone dimensions, layouts, and form factors may vary greatly, the adapter 100 may be adjustable in order to mount to various smartphones 200 in a secure manner while aligning the lens assembly 105 with the camera lens 205 as described above, irrespective of the location and/or orientation of the camera lens 205 on the body of the smartphone 201. Accordingly, the adapter 100 may be capable of use with a variety of smartphones 200 and may be referred to as a “universal” adapter.

Referring now to FIG. 3, the support 110 and the mounting assembly 115 are discussed in greater detail. As shown, the support 110 may comprise a flange or lip configured to receive a portion of the smartphone 201, e.g., an edge thereof, to stabilize the smartphone 201 in contact with the adapter 100. The mounting assembly 115 may be configured to clamp the smartphone against the body of the adapter 100 in its position upon the support 110, thereby securing the adapter 100 on the smartphone 201 in the desired position and orientation. In some embodiments, the mounting assembly 115 comprises one or more mounting arms 125 and a fixation mechanism 130. In some embodiments, the mounting assembly comprises a plurality of mounting arms 125, e.g., a first mounting arm 125A and a second mounting arm 125B, that may be positioned on opposing sides of the smartphone 201. For example, the mounting arms 125A/125B may be positioned on the lateral sides of the smartphone 201. In some embodiments, one or more of the mounting arms 125 comprise at least one gripping feature 135, e.g., a lip, a flange, a bumper, and/or the like, for contacting and/or gripping a lateral side of the smartphone. For example, the gripping features 135 may abut the sides of the smartphone 201 to grip the smartphone 201 by friction or interference. In another example, the gripping features 135 may comprise a hooking portion and/or additional features configured to securely hold the smartphone 201.

In some embodiments, the mounting arms 125 may be adjustable to change a distance between the gripping features 135 and/or the distal ends of the mounting arms 125. Accordingly, the mounting arms 125 may be moved to adjust the space therebetween in order to securely fit the smartphone 201 therebetween. For example, it may be advantageous to adjust the space between the mounting arms 125 to a size at least equal to a width of the smartphone 201 yet small enough to grip the smartphone 201 at each side to securely hold the smartphone 201 to the retinal imaging adapter 100.

In some embodiments, the mounting arms 125 may additionally or alternatively be adjustable to change a position of the mounting arms 125 with respect to the lens assembly 105. For example, it may be necessary to adjust the mounting arms 125A/125B such that, when the smartphone 201 is gripped between the mounting arms 125A/125B, the lens assembly 105 (partially depicted in FIGS. 2A and 3) is laterally aligned with the camera lens 205 of the smartphone 201. Such adjustments may be necessary to account for the varying dimensions, layouts, and form factors of different smartphones 200. Accordingly, the mounting arms 125A/125B may be laterally adjusted with respect to the lens assembly 105 to effectively change the alignment of the smartphone 201 with the lens assembly 105, thereby enabling lateral alignment of the lens assembly 105 with the camera lens 205.

In some embodiments, the mounting assembly 115 further comprises a housing 140 for the mounting arms 125A/125B. As shown in FIG. 3, the housing 140 may be configured to receive the mounting arms 125A/125B within a channel therein. Accordingly, an orientation of the mounting arms 125A/125B may be maintained while permitting lateral movement relative to one another. It should be understood that the housing 140 may be formed in a variety of manners to accomplish the described function as would be apparent to a person having an ordinary level of skill in the art.

In some embodiments, the fixation mechanism 130 may be configured to fix a position of the mounting arms 125 with respect to one another. For example, as shown in FIGS. 2A and 3, the fixation mechanism may be a fastener such as a screw. The fastener may comprise a threaded rod and a knob coupled thereto. The threaded rod may be inserted through openings in the mounting arms 125 and the knob may be turned by hand to advance the threaded rod through the openings. The threaded rod may be advanced to clamp the mounting arms 125 in a set position to securely hold the smartphone 201. For example, the threaded rod may extend through holes in the mounting arms 125 and clamp the mounting arms 125 between the knob and the housing 140. However, it should be understood that fixation of the mounting arms 125 may be accomplished in various manners as would be apparent to a person having an ordinary level of skill in the art.

In some embodiments, the support 110 may be adjustable to change a position of the support 110 with respect to the lens assembly 105. For example, it may be necessary to adjust the support 110 such that, when the smartphone 201 is rested on the support 110, the lens assembly 105 (partially depicted in FIGS. 2A and 3) is aligned with the camera lens 205 of the smartphone 201. Such adjustments may be necessary to account for the varying dimensions, layouts, and form factors of different smartphones 200. Accordingly, the support 110 may be vertically adjusted with respect to the lens assembly 105 to effectively change the alignment of the smartphone 201 with the lens assembly 105, thereby enabling alignment of the lens assembly 105 with the camera lens 205. As shown in FIG. 3, the support 110 may comprise an arm extending towards the mounting arms 125A/125B and comprising an opening for receiving the threaded rod of the fixation mechanism 130. Accordingly, the support 110 may be vertically adjusted and fixed by the fixation mechanism in the manner described above with respect to the mounting arms 125. In some embodiments, the arm of the support 110 may be received within a channel of the housing 140 in order to maintain an orientation of the support 110 with respect to the lens assembly 105 and/or the mounting arms 125 while permitting vertical movement thereof. However, it should be understood that the orientation of the support 110 may be maintained in a variety of manners as would be apparent to a person having an ordinary level of skill in the art.

In some embodiments, the mounting assembly 115 comprises a return mechanism for biasing the mounting arms 125 towards an initial arrangement. For example, the return mechanism may comprise a spring mechanism or another mechanism for biasing the mounting arms 125 to a “closed” arrangement, i.e., wherein the gripping features 135 and/or distal ends of the mounting arms 125 are relatively constricted and/or close to one another. Accordingly, a user may adjust the mounting arms 125 to an “open” arrangement, i.e., moving the gripping features 135 and/or distal ends away from one another, in order to insert the smartphone 201 therebetween. Thereafter, the mounting arms 125 may return from the open arrangement towards the closed position by the return mechanism until the mounting arms 125 contact the edges of the smartphone 201. Accordingly, the mounting assembly 115 may secure the smartphone 201 to the retinal imaging adapter 100. In some embodiments, the support 110 may be biased in a similar manner by the same return mechanism and/or an additional return mechanism. It should be understood that the return mechanism as described may be utilized in place of and/or in addition to the fixation mechanism 140 as described herein.

In some embodiments, the mounting assembly 115 may comprise more than two mounting arms 125. For example, the mounting assembly 115 may comprise three, four, or more mounting arms 125. In some embodiments, these additional mounting arms 125 are arranged and configured to contact the lateral sides of the smartphone 201. In some embodiments, the additional mountings arms 125 are arranged and configured to contact additional sides of the smartphone 201, e.g., upper and/or lower sides. It should also be understood that while the support 110 is described and depicted herein as separate from the mounting arms 125, in some embodiments the support 110 may comprise an additional mounting arm 125.

Referring once again to FIG. 5, the lens assembly 105 of the retinal imaging adapter 100 is described in greater detail. The lens assembly comprises a proximal end 145, a distal end 150, one or more condensing lenses 155, and a fiber optic tube 160. In some embodiments, the lens assembly 105 may comprise additional components, e.g., filters, condensers, diffusers, and/or additional camera components as would be known to a person having an ordinary level of skill in the art.

In some embodiments, the proximal end 145 is configured to contact a smartphone 201. For example, the proximal end 145 may interface with a surface of the smartphone 201 comprising the camera lens 205 as shown in FIGS. 2A-2B. In some embodiments, the proximal end 145 may also interface with a surface of the smartphone 201 comprising a flash unit 210 as shown in FIG. 5. The flash unit 210 may be configured to generate bright light for illuminating an eye 10 of a patient. In some embodiments, the flash unit 210 provides a short burst of light in the manner of a camera flash. In some embodiments, the flash unit 210 provides an extended period of light in the manner of a smartphone flashlight function. The flash unit 210 may be an LED light source or any other light source known to be used on smartphones as would be understood to a person having an ordinary level of skill in the art.

In some embodiments, the distal end 150 is configured to be positioned proximate an eye 10 of a patient. In some embodiments, the distal end 150 may be positioned with respect to the eye 10 such that the ophthalmoscope 100 is positioned with respect to the eye 10 in the conventional manner for an ophthalmoscope. In some embodiments, the ophthalmoscope 100 including the distal end 150 is positioned such that the central optical axis 120 is aligned with the pupil of the eye 10. However, additional arrangements are contemplated herein according to the desired type of image and desired vantage point to be captured using the retinal imaging device 200 as would be known to a person having an ordinary level of skill in the art. In some embodiments, the distal end 150 may comprise a hood portion configured to contact the patient to stabilize the ophthalmoscope 100 with respect to the eye 10 while maintaining the remainder of the lens assembly 105 at an appropriate distance from the eye 10.

In some embodiments, the condensing lenses 155 are arranged between the proximal end 145 and the distal end 150. In some embodiments, the condensing lenses 155 are arranged in series to form an imaging light pathway. In some embodiments, the condensing lenses 155 forming the imaging light pathway are aligned with the central optical axis 120 and are thus aligned with the camera lens 205. The imaging light pathway extending through the condensing lenses 155 between the proximal end 145 and the distal end 150 may be substantially translucent and/or transparent such that light may pass therebetween. Accordingly, light passing through the lens assembly 105 may be modified by diffraction through the imaging light pathway prior to reaching the camera lens 205. Furthermore, the imaging light pathway may be closed off from an external environment at the proximal end 145. For example, the lens assembly 105 may be sized configured to align at least a portion of the imaging light pathway with the camera lens 205 while the remainder of the cross-section of the imaging light pathway is covered by the surrounding surface of the smartphone 201, thereby sealing the imaging light pathway from external light. Accordingly, the camera lens 205 may receive images of the retina of the eye 10 through the imaging light pathway as modified by the lens assembly 105.

In some embodiments, the fiber optic tube 160 may extend from the proximal end 145 through the lens assembly 105 towards the distal end 150. In some embodiments, the fiber optic tube 160 extends the entire length of the lens assembly 105 from the proximal end 145 to the distal end 150. However, in additional embodiments, the fiber optic tube 160 extends a portion of the length of the lens assembly 105 as shown in FIG. 5.

The fiber optic tube 160 may comprise a donut- or ring-shaped channel. For example, as shown in FIG. 5, the fiber optic tube 160 may be bound by inner and outer circumferential surfaces with a central opening defined by the inner circumferential surface. In some embodiments, the fiber optic tube 160 is aligned with the central optical axis 120 such that the central optical axis 120 extends through the central opening. In some embodiments, the fiber optic tube 160 is arranged in a radially symmetrical manner about the central optical axis 120.

In some embodiments, the fiber optic tube 160 is aligned with the flash unit 210 at the proximal end 145 of the lens assembly 105. For example, the proximal end 145 may interface with a surface of the smartphone 201 comprising the flash unit 210 as shown in FIG. 5 such that the flash unit 210 is aligned with a portion the ring-shaped channel. Furthermore, the donut- or ring-shaped channel may be open at proximal and distal ends of the fiber optic tube 160, thereby forming an illuminating light pathway therethrough. In some embodiments, the proximal end of the fiber optic tube 160 has a proximal opening configured to be aligned with the flash unit 210 and closed off from an external environment. For example, the proximal opening may be sized configured to align with and/or receive the flash unit 210 at a portion thereof while the remainder of the proximal opening is covered by the surrounding surface of the smartphone 201, thereby sealing the proximal end of the fiber optic tube 160 from external light. In some embodiments, the proximal opening has a diameter of about 33 mm. However, the proximal opening may be sized with various diameters, e.g., about 10 mm, about 20 mm, about 30 mm, about 40 mm, about 50 mm, greater than about 50 mm, or individual values or ranges therebetween. In some embodiments, the flash unit 210 and the fiber optic tube 160 are arranged and configured such that a majority of the illuminating light from the flash unit 210 is directed into the ring-shaped channel of the fiber optic tube 160. In some embodiments, the flash unit 210 and the fiber optic tube 160 are arranged and configured such that all or substantially all of the illuminating light from the flash unit 210 is directed through the proximal opening and into the ring-shaped channel of the fiber optic tube 160.

In some embodiments, at least a portion of the distal end of the fiber optic tube 160 may be open to allow illuminating light to pass therethrough. In some embodiments, the fiber optic tube 160 comprises a distal opening extending about the entire circumference of the distal end of the ring-shaped channel. In some embodiments, the fiber optic tube 160 comprises a distal opening extending over the entire cross-section of the distal end of the ring-shaped channel. Accordingly, illuminating light from the flash unit 210 may be directed through the illuminating light pathway through the ring-shaped channel of the fiber optic tube 160 and out of the distal opening to illuminate the eye 10 for imaging. In some embodiments, the illuminating light from the fiber optic tube 160 may pass through the condensing lenses 155 to illuminate the eye 10 (see FIG. 5). In additional embodiments, the illuminating light from the fiber optic tube 160 may illuminate the eye 10 without passing through the condensing lenses 155.

The fiber optic tube 160 may comprise the components and properties of an optical fiber as would be apparent to a person having an ordinary level of skill in the art. For example, the fiber optic tube 160 may comprise a core, a cladding, and/or an outer coating. The core, cladding, and/or outer coating maybe formed using conventional materials as would be known to a person having an ordinary level of skill in the art. Accordingly, illuminating light received from the flash unit 210 may be transmitted according to the principle of total internal reflection across the illuminating light pathway from the proximal end to the distal end of the fiber optic tube 160 to illuminate the eye 10.

In some embodiments, the distal opening of the fiber optic tube 160 is covered by a diffuser element configured to diffuse the illuminating light exiting the illuminating light pathway. Accordingly, the illuminating light reaching the eye 10 may be evenly distributed and sharp points and/or shadows may be significantly reduced and/or eliminated as compared to direct light from a point source (e.g., directly from the flash unit 210).

As described herein, using the retinal imaging device 200, illuminating light may be delivered through an illuminating light pathway to illuminate the eye 10 and images of the eye 10 may be captured via an imaging light pathway by a camera lens 205. In some embodiments, the angle of delivery of illuminating light and the angle of capture of imaging light may be selected to improve the quality of the captured images. For example, the illuminating light may cause glares or reflections in captured images that may obscure portions of the images of the eye 10. Accordingly, the angle of delivery and the angle of capture may be selected according to Gullstrand's principle in order to reduce and/or eliminate such glares or reflections. According to Gullstrand's principle, the illuminating beam and the viewing beam must be totally separated through the cornea, the pupillary aperture, and the lens to avoid reflections. Furthermore, the illuminating beam and the viewing beam must coincide at the retina to permit viewing. As such, the retinal imaging adapter 100 may be structured and configured to provide an angle of delivery and an angle of capture that facilitate compliance with Gullstrand's principle over a useful range of distances of the retinal imaging device 200 from the eye. For example, the angle of delivery and angle of capture may be set in the retinal imaging adapter 100 at angles that enable separation of the illuminating beam and viewing beam through the cornea, the pupillary aperture, and the lens, and coincidence of the illuminating beam and the viewing beam at the retina when the retinal imaging device 200 is held at one or more distances from the eye 10 that are common and/or useful for retinal imaging. In some embodiments, the retinal imaging device 200 may be positioned during use at an appropriate distance from the eye 10 that facilitates compliance with Gullstrand's principle as described herein, i.e., a distance wherein the illuminating beam and viewing beam are separated through the cornea, the pupillary aperture, and the lens, and coincident at the retina. Accordingly, the retinal imaging device 200 may advantageous capture clear and unobscured images of the retina. In some embodiments, the captured image of the retina may cover a larger region of the retina than conventional devices are able to capture.

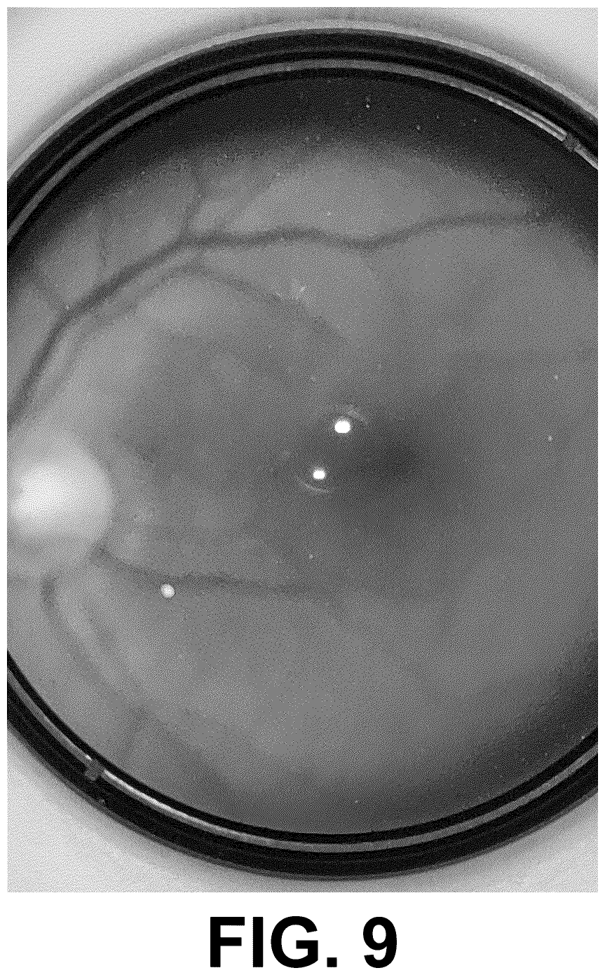

Additionally, the retinal imaging device 200 may provide improved illumination that enables collection of improved images of the retina. As described herein, the illuminating light pathway delivers light in a ring-shaped profile through the fiber optic tube 160 and may be diffused through a diffuser element. The diffuser element and/or the ring-shaped profile reduces the occurrence of “hot spots” from the light source. For example, while light from a typical light source may be more intense at the center than at the periphery, the light transmitted from the fiber optic tube 160 delivers light through a diffuser element and in a ring-shaped profile without light emission at the center, thereby producing a more even and consistent intensity of light. In some embodiments, the described configured reduces the overall intensity of the illuminating light. Furthermore, in some embodiments, the lens assembly 105 may comprise additional filters or other components in the illuminating light pathway to reduce the intensity of the illuminating light. Advantageously, the less intense and/or diffused light delivered by the retinal imaging device 200 allows the pupil of the eye 10 to remain dilated to a greater degree than is typically possible using conventional fundus camera, e.g., providing direct light from a point source. The greater dilation of the pupil results in clearer images of the retina comprising larger and/or more visible features. Accordingly, the retinal imaging device 200 as described herein may provide images of the retina with greater clarity that may facilitate easier assessment and/or diagnosis of the eye 10 as further described herein. FIG. 9 depicts an exemplary retinal image captured using the devices, systems, and methods described herein.

The devices, systems, and methods as described herein are not intended to be limited in terms of the particular embodiments described, which are intended only as illustrations of various features. Many modifications and variations to the devices, systems, and methods can be made without departing from their spirit and scope, as will be apparent to those skilled in the art.

While the retinal imaging adapter 100 is described and depicted for use with a smartphone 200, it should be understood that that retinal imaging adapter 100 may be applied to additional devices comprising a camera and/or configured to hold such a device, e.g., a tablet, a laptop computer, an action camera (e.g., a GoPro device), a camera mount or stabilizer (e.g., a DJI Osmo device), a virtual reality headset, an augmented reality headset, a drone, and additional types of devices as would be known to a person having an ordinary level of skill in the art.

### System for Processing a Retinal Image

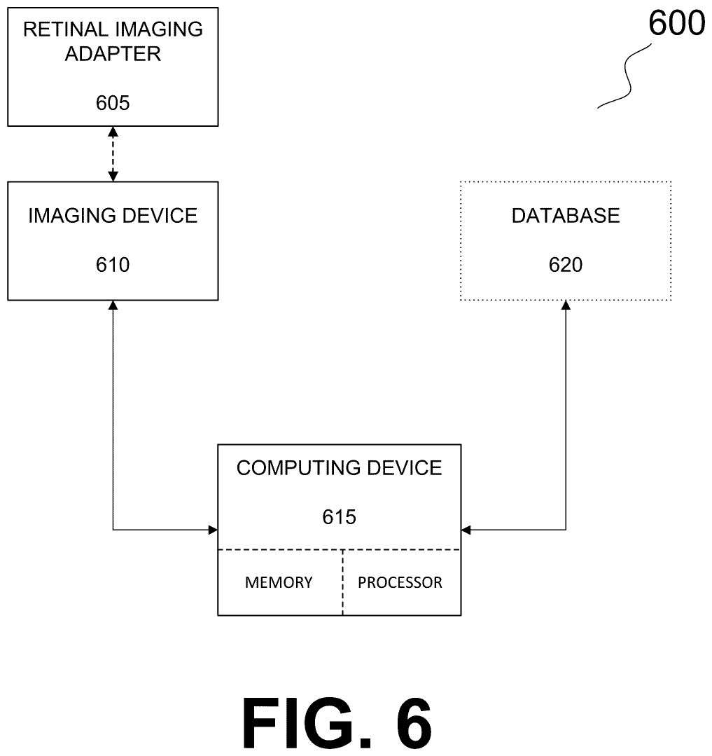

Referring now to FIG. 6, a block diagram of an exemplary system for processing a retinal image (e.g., a fundus image) is depicted in accordance with an embodiment. The system 600 comprises a retinal imaging adapter 605, an imaging device 610, and a computing device 615. The retinal imaging adapter 605 and the imaging device 610 may be in optical communication and/or optically aligned with one another and configured to be optically aligned with an eye 10 of a patient. The imaging device 610 may be in electrical communication with the computing device 615. In some embodiments, the system 600 further comprises an external database 620 in operable communication with the computing device 615.

The retinal imaging adapter 605 may comprise a lens assembly, a support, and/or a mounting assembly. It should be understood that the retinal imaging adapter 605 may comprise an ophthalmoscope such as the retinal imaging adapter 100 as described herein with respect to FIGS. 1-5 and may comprise any of the features and/or functions as described with respect to the retinal imaging adapter 100.

The imaging device 610 may comprise a camera and/or another type of imaging device having a camera lens. In some embodiments, the imaging device 610 comprises a mobile device having a camera. It should be understood that the imaging device 610 may comprise a smartphone such as the smartphone 201 as described herein with respect to FIGS. 1-5 and may comprise any of the features and/or functions as described with respect to the smartphone 201. Further, the imaging device 610 may comprise other types of devices having some or all of the described features and/or functions, e.g., a tablet, a laptop computer, an action camera (e.g., a GoPro device), a camera mount or stabilizer (e.g., a DJI Osmo device), a virtual reality headset, an augmented reality headset, a drone, and additional types of devices as would be known to a person having an ordinary level of skill in the art.

In some embodiments, the imaging device 610 and the retinal imaging adapter 605 may be in optical communication. For example, a camera lens of the imaging device 610 may be aligned with an end of the lens assembly of the retinal imaging adapter 605 such that an optical axis of the camera lens of the imaging device 610 is coincident or co-extensive with an optical axis of the lens assembly of retinal imaging adapter 605 (e.g., as shown in FIG. 5). As such, light reaching the camera lens of the imaging device 610 may be modified by diffraction through the lens assembly of retinal imaging adapter 605 and thus images obtained by the imaging device 610 may be modified by the retinal imaging adapter 605. Accordingly, the assembled imaging device 610 and retinal imaging adapter 605 may be aligned with an eye 10 of a patient in order to capture images thereof as described herein. As further described herein, when the imaging device 610 is assembled with the ophthalmoscope 605, the assembly may form a digital fundus camera and thus the assembly may be interchangeably referred to as a digital fundus camera throughout this disclosure.

The computing device 615 may be in electrical communication with the imaging device 610 in order to receive images of the eye 10 collected by the imaging device 610 and process the images as further described herein. In some embodiments, the computing device 615 includes a processor and a memory such as a non-transitory, computer-readable medium storing instructions for processing retinal images. It should be understood that the computing device 615 may comprise any number of components of a computing device as would be known to a person having an ordinary level of skill in the art for communication, processing, and powering the system 600. In some embodiments, the computing device 615 may be a processor and/or memory of a smartphone. For example, a smartphone may comprise the imaging device 610 and the computing device 615. Accordingly, a software application on the smartphone may be executed to receive images of the eye 10 and process the images as further described herein. In additional embodiments, the computing device 615 may be an external device that is not housed with the imaging device 610. Accordingly, a communications unit (e.g., wired or wireless communication) may be used to transmit images collected by the imaging device 610 to the computing device 615 for processing.

Referring once again to FIG. 6, in some embodiments, the system 600 may further comprise a database 620. For example, the computing device 615 may access a database 620 comprising information related to features and/or patterns associated with various medical states and/or symptoms thereof associated with the eye, e.g., the retina. In some embodiments, the database 620 may be stored on the memory of a smartphone (e.g., the smartphone housing the imaging device 610 and/or the computing device 615) or another location accessible to the computing device 615, e.g., a remote database, cloud server, or another external computing device. The computing device 615 may compare the captured images from the imaging device 610 to the information in the database 620 in order to process the images. For example, the computing device 615 may identify features in the images and/or provide information related to a diagnosis for the eye 10.

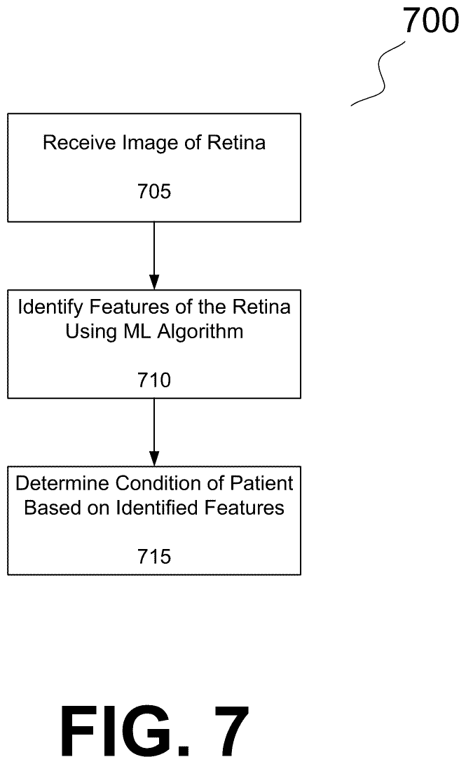

Referring now to FIG. 7, an illustrative method of processing a retinal image by the system 600 is depicted in accordance with an embodiment. For example, the method 700 may be carried out by the processor of the computing device 615 upon execution of the instructions stored on the memory. The method 700 comprises receiving 705 an image of a retina of a patient, identifying 710 one or more features of the retina using a machine learning algorithm based on the image, and determining 715 a condition of the patient based on the one or more identified features. In some embodiments, the condition of the patient may relate to the health of the patient, a disease state of the patient, and/or a disease stage of the patient. In some embodiments, the condition of the patient relates to the eye and/or ocular tissue of the patient. In some embodiments, the condition of the patient relates to the retina of the patient.

In some embodiments, the image is obtained using the retinal imaging adapter 605 and the imaging device 610 (i.e., the digital fundus camera) of the system 600 and may be received by the computing device 615 in the manner described herein. It should be understood that the computing device 615 may receive 705 a plurality of images of the retina in this manner. In some embodiments, the method 700 is performed using a plurality of images of the retina.

In some embodiments, identifying 710 one or more features of the retina comprises processing the image using the machine learning algorithm. In some embodiments, the machine learning algorithm may be in electrical communication with a database of medical data (e.g., ophthalmological data). In some embodiments, the database is a database 620 as described herein with respect to FIG. 6 and may include any of the various types of information discloses with respect to the database 620. The ophthalmological data may be accessed from the database 620 to process the image and/or to identify 710 one or more features in the image. In some embodiments, the database 620 stores information related features of interest, e.g., sample signals, patterns, characteristics, and the like that may be identified 710 in images of the retina. In some embodiments, the ophthalmological data comprises one or more known ophthalmological features. For example, the database 620 may comprise a library of known ophthalmological features. The known ophthalmological features may be documented and/or known to be associated with particular conditions or states of the patient, e.g., through research and/or assessment of images from historical patients. In some embodiments, the database 620 further comprises a library of images from historical patients, which may be used for training and/or comparison by the machine learning algorithm. Accordingly, detecting the known ophthalmological signatures may be indicative of particular conditions, i.e., diagnoses. In some embodiments, the computing device 615 may access a subset of the library based on known patient parameters (e.g., gender, age, medical history, and the like) such that the computing device 615 may assess the image based on similar patients using known patient parameters. In some embodiments, the computing device may communicate with the database 620 remotely and access the ophthalmological data externally from the computing device 615, e.g., at a remote computer, server, cloud database, and/or the like. In some embodiments, the computing device 615 may download and/or store the ophthalmological data on the computing device 615, e.g., on the memory. Accordingly, the method 700 may be performed entirely “offline,” i.e., without access to any remote database.

The computing device 615 may identify 710 one or more features using the machine learning algorithm in a variety of manners as would be known to a person having an ordinary level of skill in the art. In some embodiments, the machine learning algorithm, may identify 710 the one or more features by comparison to the ophthalmological data, e.g., known ophthalmological features, to identify the one or more features in the image. In some embodiments, the database 620 may include information or details to enable the machine learning algorithm to identify the one or more features.

In some embodiments, the computing device 615 may process the images to detect one or more features in the image. In some embodiments, the one or more features may comprise anatomical structures of the retina, characteristics of the eye and/or the retina, and the like. In some embodiments, the one or more features include a color of the eye. In some embodiments, the one or more features include vessels within the eye and/or retina. In some embodiments, the one or more features include categories of vessels, e.g., particular shapes. In some embodiments, the images may be labeled or notated to mark the one or more identified features on the image. In some embodiments, additional measurements or calculations may be performed on the image related to the one or more identified features. For example, a size, shape, location, and/or proximity of an identified feature with respect to an anatomical structure and/or another identified feature may be determined based on the image. The additional measurements and/or calculations may be notated on the image. Accordingly, the processed image may facilitate diagnosis by a medical professional (e.g., an ophthalmologist) viewing the image as further described herein.

In some embodiments, the computing device 615 may determine 715 a condition of the patient based on the one or more identified features. For example, the identification of a known ophthalmological feature may, in some instances, indicate a particular condition of the patient. Accordingly, the computing device 615 may determine the condition of the patient based on the one or more identified features. In additional embodiments, the computing device 615 may rely on one or more calculations and/or ophthalmological data in the database 620 to determine a condition of the patient.

In some embodiments, the computing device 615 may determine 715 the condition of the patient based on user input. For example, the processed image, i.e., the image demonstrating one or more identified features and information related thereto, may be displayed to a user (e.g., a medical professional such as an ophthalmologist). The processed image may be viewed by the user and the user may provide input via an input device to indicate a condition of the patient as further described herein. In some embodiments, the user may view the processed image on a display within the system 600 (e.g., a display of the imaging device 610 such as a smartphone) and input may be provided through input features on the imaging device 610. In some embodiments, the processed image may be transmitted to remote device for viewing and input from an ophthalmologist and/or other medical professional. For example, the processed image may be transmitted via internet. Accordingly, the computing device 615 may receive input remotely in order to determine 715 the condition of the patient. In some embodiments, the input comprises a diagnosis of the condition of the patient. In additional embodiments, the computing device may make an initial determination (i.e., a prediction) without user input as described above, and the processed image and the prediction may then be shared with a user. In some embodiments, the prediction may include a probability associated therewith as described herein. Accordingly, the input may comprise confirmation of the prediction, rejection of the prediction, and/or a new diagnosis. In some embodiments, the user may be presented with a plurality of predictions as described herein. In some embodiments, each prediction may include a probability associated therewith as described herein. Accordingly, the input may comprise confirmation of one of the predictions, rejection of one or more of the predictions, and/or a new diagnosis not among the predictions.

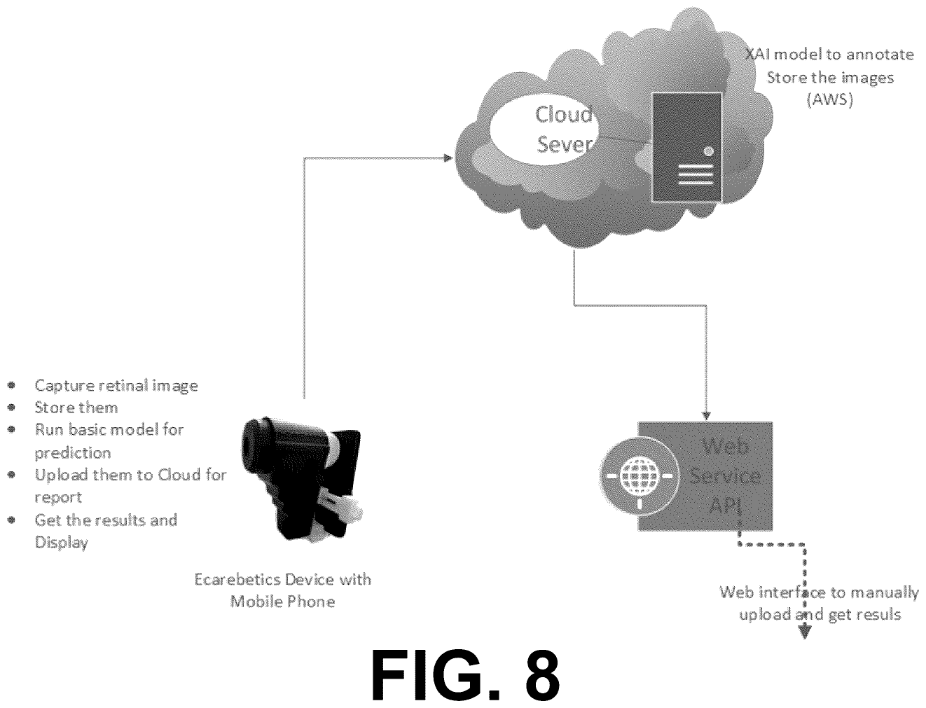

In some embodiments, the computing device 615 determines 715 the condition of the eye in substantially real time. In some embodiments, the computing device 615 may alternatively operate in both an online and an offline mode. For example, if internet is not available and/or connected, the determination 715 may occur on the computing device 615 (i.e., offline mode) without input from a user or another external source. Furthermore, if internet is available and connected, the determination 715 may occur with input from a user or another external source as described (i.e., online mode). Accordingly, the processed images may be uploaded or transmitted externally to obtain interpretation and/or feedback. FIG. 8 depicts a network within which the system 600 operates in accordance with an embodiment. For example, the system 600 may be embodied in a smartphone comprising a mobile application as shown.

In some embodiments, the condition of the patient may relate to health, a disease state, and/or a disease stage of the patient. In some embodiments, the condition of the patient relates to the eye and/or ocular tissue of the patient. In some embodiments, the condition of the patient relates to the retina of the patient. In a particular example, the condition may relation to diabetic retinopathy. In some embodiments, the computing device 615 determines 715 whether the patient has diabetic retinopathy. In some embodiments, the computing device 615 determines 715 a stage of diabetic retinopathy in the patient. For example, the computing device 615 may classify the retina based on known stages that are commonly used and understood in the field of ophthalmology with respect to diabetic retinopathy. In some embodiments, the computing device 615 may classify the retina into one of five stages: normal, mild, moderate, severe, and proliferative diabetic retinopathy.

In some embodiments, the determination 715 may include a probability. For example, the computing device 615 may output a diagnosis and a probability of accuracy of the diagnosis. In some embodiments, the determination 715 may include a plurality of diagnoses, wherein each diagnosis has an associated probability. In some embodiments, the sum of the probabilities of the plurality of diagnoses is 1 or 100%.

In some embodiments, the method further comprises outputting a follow-up instruction for the patient. In some embodiments, the follow-up instruction comprises a treatment. In some embodiments, the follow-up instruction comprises an instruction to consult with a medical professional.

In some embodiments, the determination 715 may be provided in the form of a report that includes one or more of the identified features, the determined condition(s), the probability associated with the determined condition(s), and follow-up instructions.

The devices, systems, and methods as described herein are not intended to be limited in terms of the particular embodiments described, which are intended only as illustrations of various features. Many modifications and variations to the devices, systems, and methods can be made without departing from their spirit and scope, as will be apparent to those skilled in the art.

While the present disclosure is discussed in detail with respect to diagnosing diabetic retinopathy, it should be understood that the system 600 and/or the method 700 may be applied to additional conditions. In some embodiments, the system 600 and/or the method 700 may be applied to identifying and/or assessing glaucoma. In some embodiments, the system 600 and/or the method 700 may be applied to identifying and/or assessing age-related macular degeneration. In some embodiments, the system 600 and/or the method 700 may be applied to identifying and/or assessing retina vein occlusion (RVO). In some embodiments, the system 600 and/or the method 700 may be applied to identifying and/or assessing retinal detachment (RD). In some embodiments, the system 600 and/or the method 700 may be applied to identifying and/or assessing fundus tumors. However, it should be understood that system 600 and/or the method 700 may be applied to additional conditions related to the retina and/or the fundus. Furthermore, while the system 600 and method 700 are described herein with respect to images of the retina and assessing conditions related to the retina, it should be understood that the system 600 and/or the method 700 may be applied to images of other types of ocular tissue (e.g., any ocular tissue that may be imaged using a fundus camera) and identifying and/or assessing conditions related thereto.

In some embodiments, the machine learning algorithm may be embodied in a convolutional neural network (CNN). In some embodiments, the machine learning algorithm may utilize a generative adversarial network (GAN) model to train and/or assess with respect to images as described herein.

As described herein, a machine learning algorithm may be utilized to identify 710 one or more features and/or determine 715 a condition of the patient. For example, a machine learning algorithm may be trained using a set of training data. In some embodiments, the set of training data comprises clinical data from historical patients, e.g., ocular images. The training data may be used to train the machine learning algorithm to identify ophthalmological features. Furthermore, the training data may be used to train the machine learning algorithm to associate identified ophthalmological features with particular conditions. For example, the training data may include outcomes from patient assessments from historical patients such that there is an indication of the accuracy of the assessment, e.g., wherein a condition of the patient was determined and/or verified by other means. Accordingly, the machine learning algorithm may become more proficient in identifying ophthalmological features and/or associating features with conditions over time. In some embodiments, a trained machine learning algorithm may further identify new and useful ophthalmological features, thereby improving the ability of the computing device 615 to identify conditions and/or expanding the variety of conditions that may be identified based on ophthalmological features. Treatment data from the historical patients may similarly be used to train the machine learning algorithm to identify effective treatments for a condition.

In some embodiments, the machine learning algorithms may be continuously trained and thus improved. For example, the machine learning algorithm may be trained using a first set of training data, which may be “seed data.” The seed data may comprise clinical data from historical patients as described herein. The seed data may be of at least a critical volume to enable the machine learning algorithm to satisfactorily identify ophthalmological features and/or determine conditions. Following the performance of the method 700, the clinical data of the patient, including demographic information, ophthalmological features, diagnoses, treatment information and/or outcome information, may be used to further train the machine learning algorithms. For example, where a particular diagnosis is rejected by a user, the machine learning algorithm may obtain an indication of these outcomes and may be trained over time to provide different and/or better predictions or proposals in similar scenarios. In another example, where a particular diagnosis received positive feedback, the machine learning algorithm may obtain an indication of these outcomes and may be trained over time to provide similar predictions or proposals in similar scenarios. Probabilities associated with identified conditions may likewise be updated and improved based on this data. In another example, after treatment is applied, the machine learning algorithm may obtain outcome data associated with the success or failure of the treatment and may be trained over time to recognize treatment parameters with a high likelihood of success and/or a low likelihood of success. Accordingly, live cases may be used to form a second set of training data, which may be “refinement data” that is used on a continual basis to re-train the machine learning algorithms.

In a specific embodiment, the system 600 and/or the method 700 utilizes a customized CNN based on DenseNet-50 for the classification of ophthalmological features. This CNN-based model is designed to accurately identify and categorize five stages of DR through the analysis of ocular images. Variational Autoencoder (VAE) is incorporated with the system as well. This VAE-based model is advantageous because it addresses the challenge of imbalanced datasets by generating additional synthetic images. By doing so, the model aims to enhance the diversity and abundance of training samples, ultimately improving the model's performance.

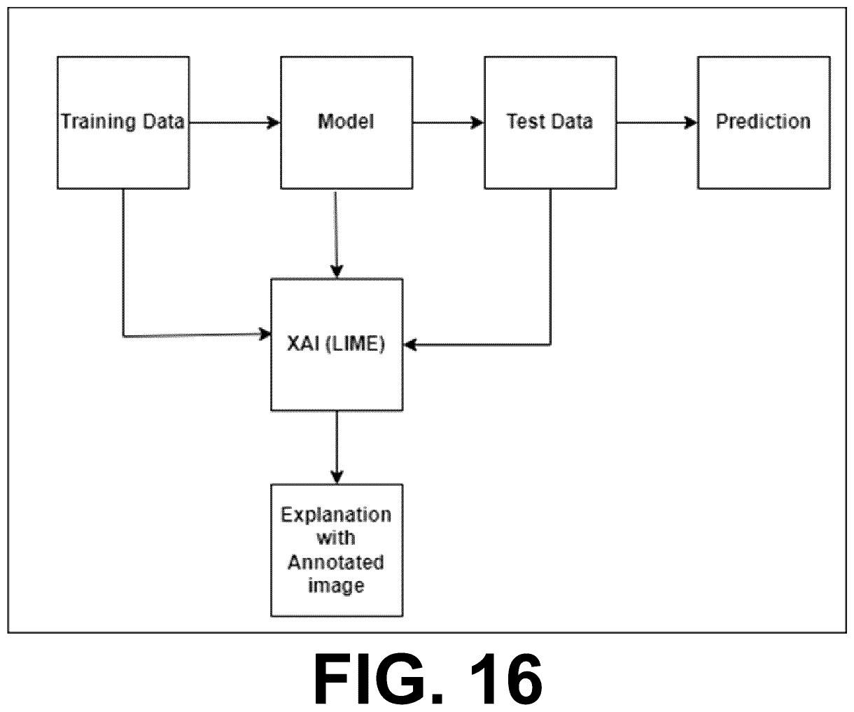

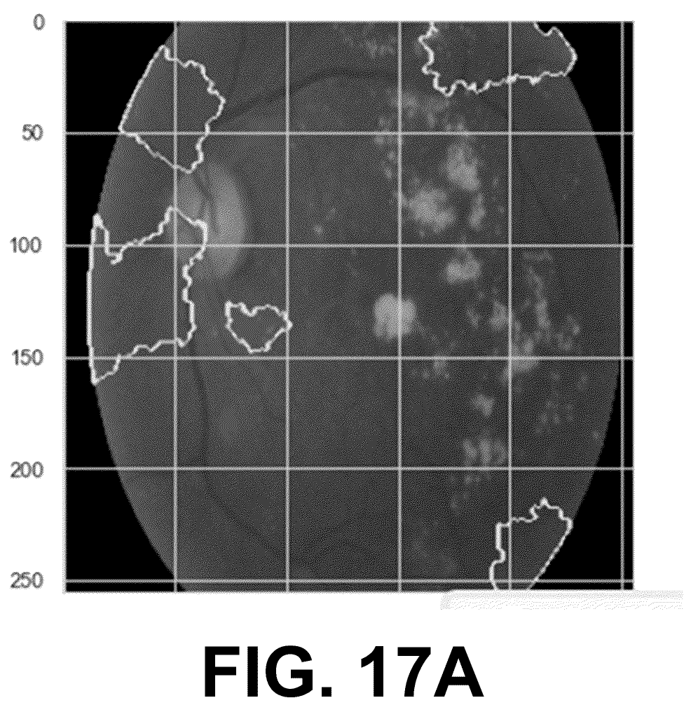

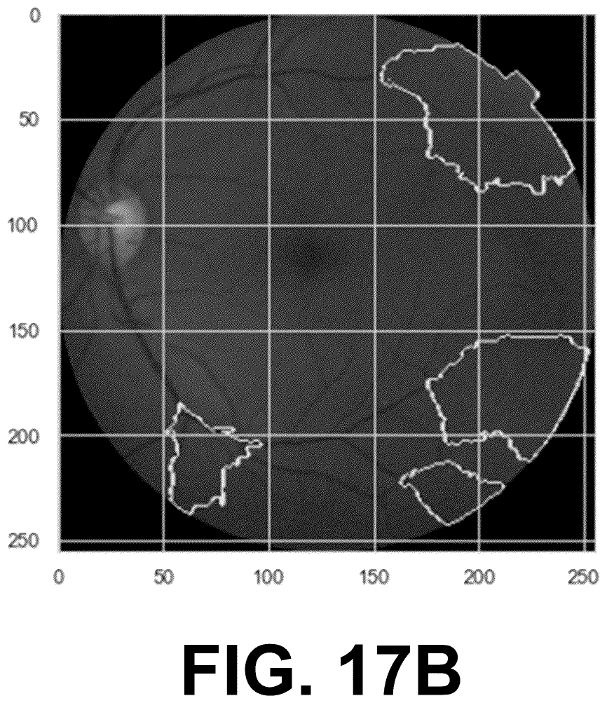

FIG. 16 depicts a LIME-based explanation framework that may be incorporated by the system 600 in the image processing and/or assessment in order to facilitate identification of specific areas within ocular images that contribute significantly to the classification results. FIGS. 17A-17B depict exemplary LIME-generated images generated through the LIME-based explanation framework that indicate areas of concerns that may be relevant to decision making. By marking these biomarker regions as illustrated in FIGS. 17A-17B, the model is more easily interpretable and may be utilized to identify ophthalmological features contributing to decision-making and/or diagnosis. Accordingly, the model provides valuable insights into the decision-making process. For example, the predicted results may be verified by this explanation and may be used to automatically generate reports along with the results. In some embodiments, a trained machine learning algorithm may further identify new and useful ophthalmological features, thereby improving the ability of the computing device 615 to identify conditions and/or expanding the variety of conditions that may be identified based on ophthalmological features.

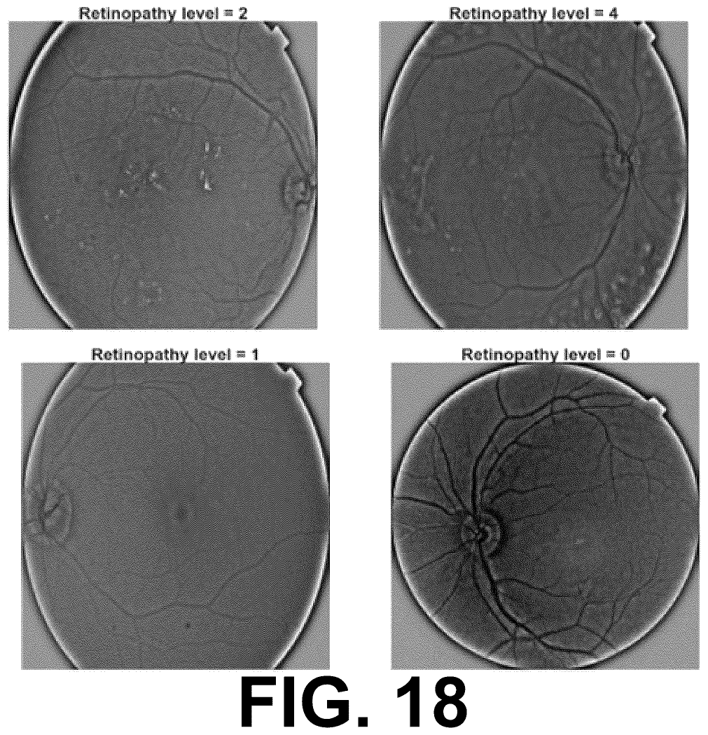

In some embodiments, the data undergoes pre-processing before being fed into the deep learning model. Pre-processing enables the execution of a five-stage classification directly on a mobile device. FIG. 18 depicts exemplary pre-processed images in accordance with some embodiments. As shown in FIG. 8, the system may function within the network to upload the pre-processed data to the cloud for the application of LIME-based explanation protocol using machine learning or artificial intelligence, thereby resulting in the generation of comprehensive reports. These reports may be easily accessible through mobile devices. Furthermore, the system may be designed to facilitate image uploads via a web API and receive predictions and detailed reports in return. The mobile-based model is trained in the cloud, and the smartphone may be utilized solely for making inferences, thereby supporting offline functionality.

In some embodiments, the model employed in the system undergoes continuous training to achieve ongoing improvements. This dynamic system facilitates perpetual model updates through the integration of live cases as refinement data. By continuously assimilating insights from new patient data, the model undergoes adaptive evolution, responding to changing conditions, and enhancing its predictive capabilities. The model's training initiates with a foundational dataset, i.e., the “seed data.” The seed data represents a critical volume, ensuring the machine learning algorithm's effective identification of ophthalmological features and determination of conditions. For instance, user rejection of a specific diagnosis prompts the model to gather feedback from healthcare professionals, enabling the model to iteratively improve predictions in similar scenarios. Conversely, positive feedback on a diagnosis empowers the model to reinforce the same predictive capabilities in analogous situations. The system continually updates and refines the probabilities associated with identified conditions based on accumulated data. Additionally, post-treatment, the model may acquire outcome data related to the treatment's success or failure. Over time, the model may learn to recognize treatment parameters with varying success probabilities. Live cases may form a dynamic second set of training data, i.e., the “refinement data.” Continual integration of refinement data ensures that the machine learning algorithms remain in a perpetual state of training, enhancing their adaptability and performance. This continual learning is processed in the cloud and mobile phone models are updated wirelessly.

The systems and methods described herein are capable of leveraging the power of transfer learning. As described, the model is initially trained using a public dataset, and this knowledge is then fine-tuned with images captured by the device on live cases. Transfer learning ensures that the model can adapt and specialize in the context of ophthalmological assessments.

### Data Processing Systems for Implementing Embodiments Herein

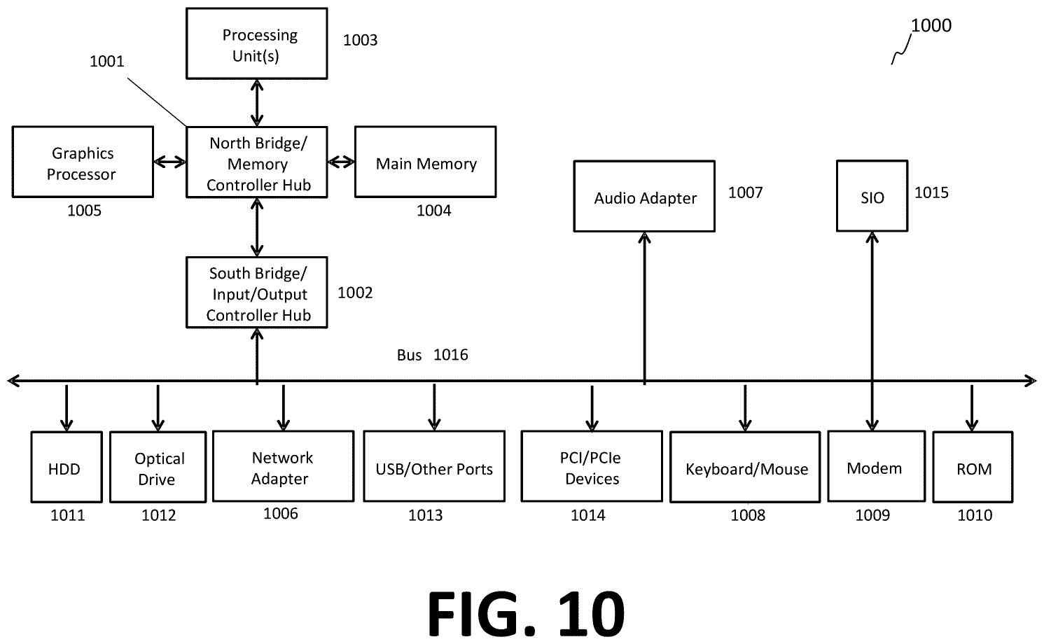

FIG. 10 illustrates a block diagram of an exemplary data processing system 1000 in which embodiments are implemented. The data processing system 1000 is an example of a computer, such as a server or client, in which computer usable code or instructions implementing the process for illustrative embodiments of the present invention are located. In some embodiments, the data processing system 1000 may be a server computing device. For example, data processing system 1000 can be implemented in a server or another similar computing device operably connected to a retinal imaging adapter 100, a digital fundus camera 200, and/or a system 600 as described above. The data processing system 1000 can be configured to, for example, transmit and receive information related to an image captured by the retinal imaging adapter 100, the digital fundus camera 200, and/or the system 600.

In the depicted example, data processing system 1000 can employ a hub architecture including a north bridge and memory controller hub (NB/MCH) 1001 and south bridge and input/output (I/O) controller hub (SB/ICH) 1002. Processing unit 1003, main memory 1004, and graphics processor 1005 can be connected to the NB/MCH 1001. Graphics processor 1005 can be connected to the NB/MCH 1001 through, for example, an accelerated graphics port (AGP).

In the depicted example, a network adapter 1006 connects to the SB/ICH 1002. An audio adapter 1007, keyboard and mouse adapter 1008, modem 1009, read only memory (ROM) 1010, hard disk drive (HDD) 1011, optical drive (e.g., CD or DVD) 1012, universal serial bus (USB) ports and other communication ports 1013, and PCI/PCIe devices 1014 may connect to the SB/ICH 1002 through bus system 1016. PCI/PCIe devices 1014 may include Ethernet adapters, add-in cards, and PC cards for notebook computers. ROM 1010 may be, for example, a flash basic input/output system (BIOS). The HDD 1011 and optical drive 1012 can use an integrated drive electronics (IDE) or serial advanced technology attachment (SATA) interface. A super I/O (SIO) device 1015 can be connected to the SB/ICH 1002.

An operating system can run on the processing unit 1003. The operating system can coordinate and provide control of various components within the data processing system 1000. As a client, the operating system can be a commercially available operating system. An object-oriented programming system, such as the Java™ programming system, may run in conjunction with the operating system and provide calls to the operating system from the object-oriented programs or applications executing on the data processing system 1000. As a server, the data processing system 1000 can be an IBM® eServer™ System® running the Advanced Interactive Executive operating system or the Linux operating system. The data processing system 1000 can be a symmetric multiprocessor (SMP) system that can include a plurality of processors in the processing unit 1003. Alternatively, a single processor system may be employed.

Instructions for the operating system, the object-oriented programming system, and applications or programs are located on storage devices, such as the HDD 1011, and are loaded into the main memory 1004 for execution by the processing unit 1003. The processes for embodiments described herein can be performed by the processing unit 1003 using computer usable program code, which can be located in a memory such as, for example, main memory 1004, ROM 1010, or in one or more peripheral devices.

A bus system 1016 can be comprised of one or more busses. The bus system 1016 can be implemented using any type of communication fabric or architecture that can provide for a transfer of data between different components or devices attached to the fabric or architecture. A communication unit such as the modem 1009 or the network adapter 1006 can include one or more devices that can be used to transmit and receive data.

Those of ordinary skill in the art will appreciate that the hardware depicted in FIG. 10 may vary depending on the implementation. Other internal hardware or peripheral devices, such as flash memory, equivalent non-volatile memory, or optical disk drives may be used in addition to or in place of the hardware depicted. Moreover, the data processing system 1000 can take the form of any of a number of different data processing systems, including but not limited to, client computing devices, server computing devices, tablet computers, laptop computers, telephone or other communication devices, personal digital assistants, and the like. Essentially, data processing system 1000 can be any known or later developed data processing system without architectural limitation.

### Additional Embodiments of Retinal Imaging Adapters and Systems Therefor

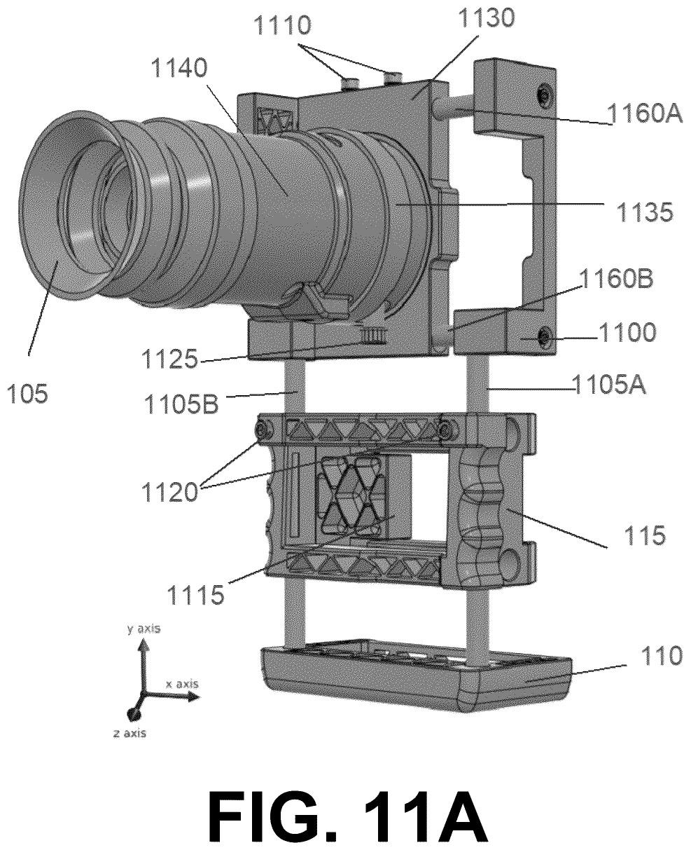

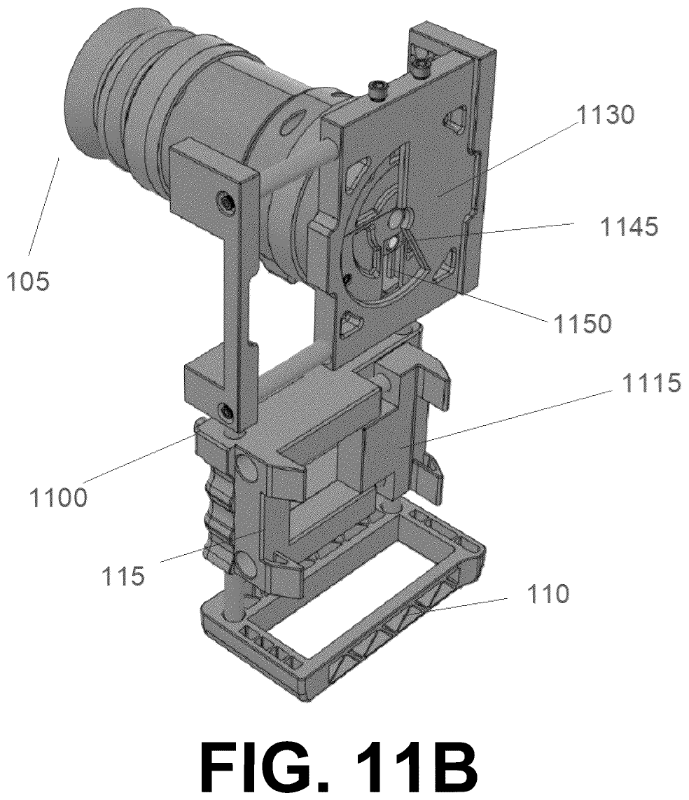

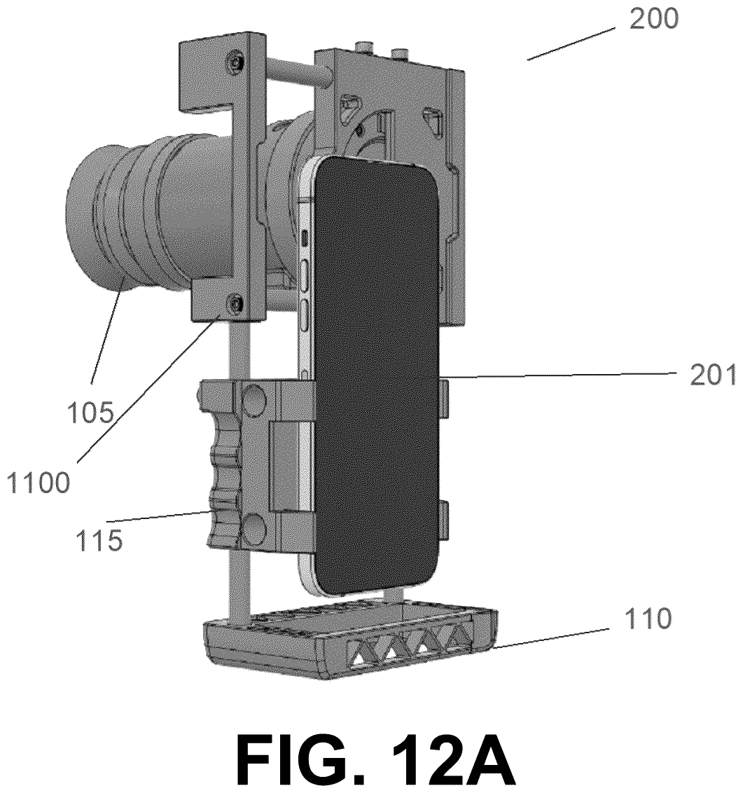

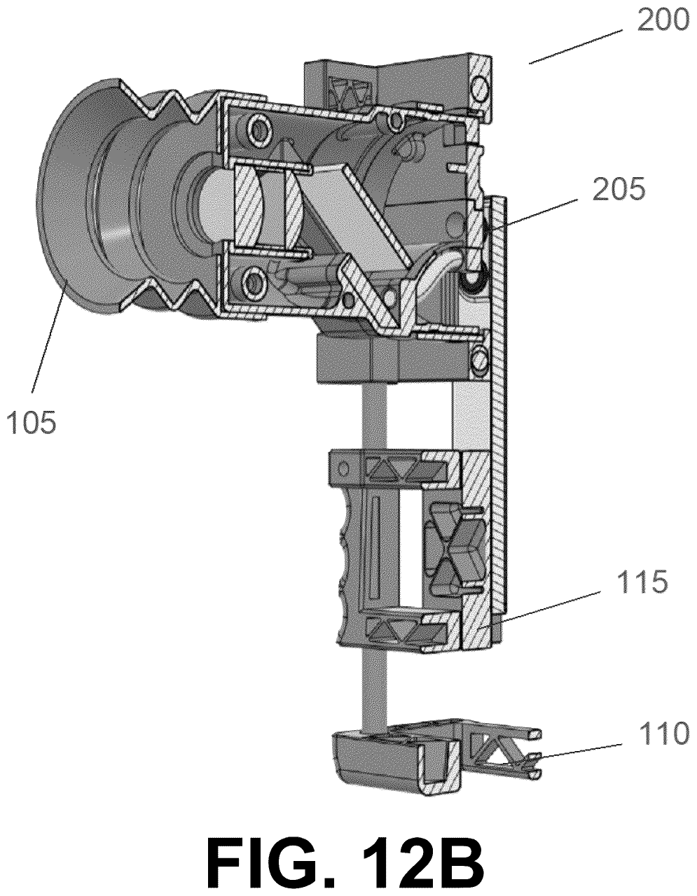

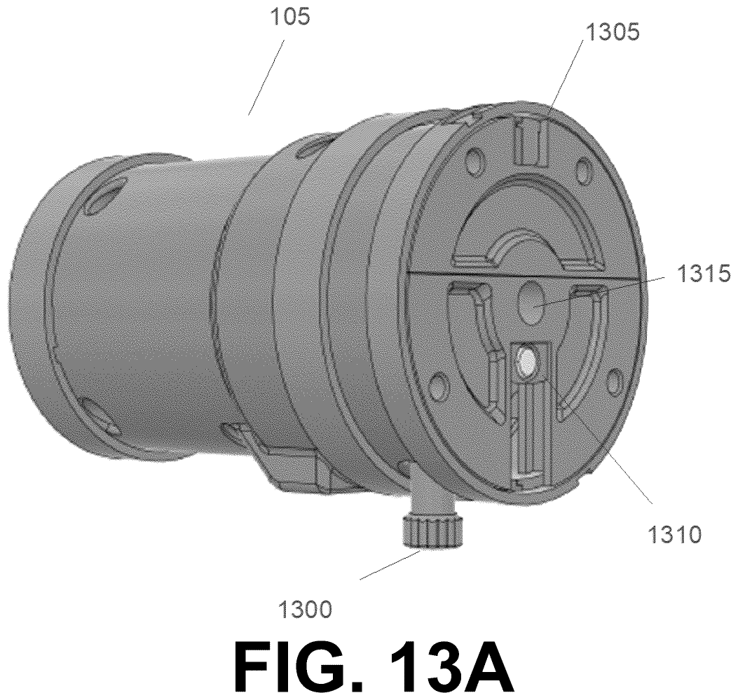

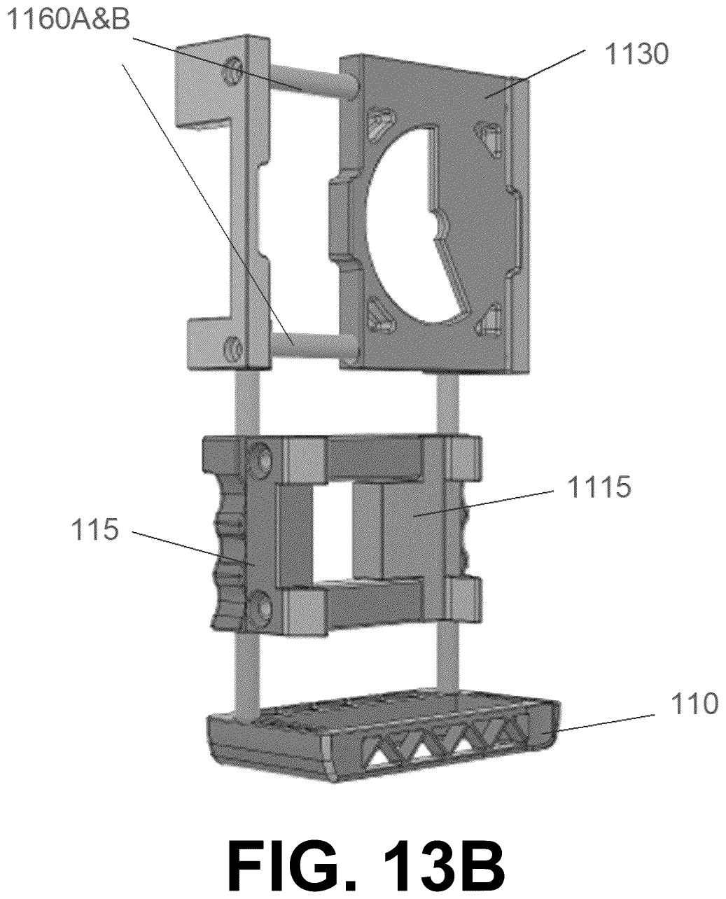

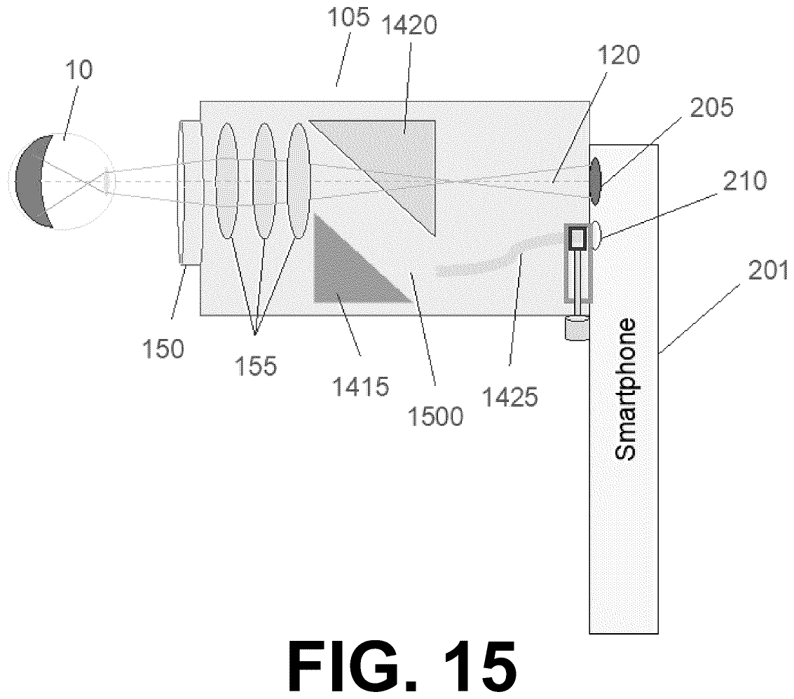

Referring now to FIGS. 11-14, an exemplary smartphone-compatible retinal imaging adapter 100 is depicted in accordance with an alternate embodiment. Furthermore, FIG. 15 depicts a retinal imaging adapter 100 assembled with a smartphone to form a retinal imaging device 200 in accordance with an alternate embodiment. It should be understood that the imaging adapter 100 and imaging device 200 as shown in FIGS. 11-15 may comprise any of the features and/or functions as described with respect to the imaging adapter 100 and imaging device 200 of FIGS. 1-5 and may be utilized as part of a system 600 as shown in FIG. 6 and/or using the method 700 of FIG. 7. The eye 10 may be the focal point of the images acquired using the retinal imaging device 200, and the imaging adapter 100 and/or the lens assembly 105 play a vital role in capturing detailed images of the retina. Similar features to the features depicted in FIGS. 1-5 may be identified herein with common reference numbers, while newly introduced features may be identified with unique reference numbers.

As discussed herein, the imaging adapter 100 may comprises a lens assembly 105, a base mount 110 to support a smartphone in an upright position, and a mounting assembly or grip 115 to securely hold the smartphone to the imaging adapter 100. In some embodiments, the lens assembly 105 includes a sophisticated arrangement of optical elements, such as multiple lenses, mirrors, and flexible waveguides as shown and further described herein. The optical elements of the lens assembly 105 may be housed within an optical housing unit 1140. The mounting assembly 115 may be sized and configured to accommodate smartphones of various sizes and/or designs. The mounting assembly 115 may comprise a spring mechanism 1115 for adjustably clamping the smartphone in place. The spring mechanism 1115 may be adjusted to securely fit a wide range of smartphone models (e.g., different sizes and/or geometries).

As shown most clearly in FIGS. 11A-11B, the mounting assembly 115 as described above may be part of and/or may further comprise an adjustable mounting device 1100 as shown most clearly in FIGS. 11A-11B and described herein. The mounting device 1100 enables a smartphone to be adjustably coupled to the lens assembly 105 with a greater degree of adjustability, thereby ensuring alignment of the optical axis of the lens assembly 105 with the camera lens 205 of the smartphone 201. This configuration integrates the imaging adapter 100 seamlessly with the smartphone in order to optimize imaging performance.

In some embodiments, the mounting device 1100 comprises a mounting plate 1130, which is configured to move horizontally along cylindrical rails 1160A/1160B. The mounting plate 1130 hosts includes a rim attachment 1135 configured to interface with the optical housing unit 1140 to connect the lens assembly 105 to the mounting plate 1130. The mounting plate 1130 may be adjusted along the cylindrical rails 1160A/1160B in order to precisely horizontally align the lens assembly 105 with a camera lens of the smartphone (e.g., the primary camera lens thereof). Accordingly, although camera lens locations may vary between smartphone models, the imaging adapter 100 may be adjusted to enable alignment of the lens assembly 105 with the camera lens of a variety of different smartphone models.

The optical housing unit 1140 is configured enable about 200 degrees of rotation of the lens assembly 105 about its longitudinal axis. For example, while coupled to the rim attachment 1135, the optical housing 1140 may rotate up to about 200 degrees to suitably align the assembly of optical elements housed therein with the camera lens 205 and the flash unit 210 of the smartphone. Particularly, the rotation capability between the optical housing unit 1140 and the rim attachment 1135 enables optimal alignment of a flexible waveguide within the lens assembly 105 with the camera lens 205 and flash unit 210. In some embodiments, the optical housing 1140 may be configured for greater than about 200 degrees of rotation, e.g., about 220 degrees, about 240 degrees, about 260 degrees, about 270 degrees, about 315 degrees, about 360 degrees, or individual values or ranges therebetween.

In some embodiments, the imaging adapter 100 comprises an adjustment knob 1125 configured to adjust a position of the flexible waveguide along a single axis. This feature ensures precise alignment of the waveguide with the flash unit 210, thereby enhancing the illumination of the images captured using the imaging adapter 100 and the resulting quality of the images. Notably, the adjustment knob 1125 may enable adjustment of the flexible waveguide independently from other optical elements in the lens assembly 105 to accommodate a variety of different smartphone models because the layout between the camera lens 205 and the flash unit 210 may vary between different smartphone models.

The imaging adapter 100 may also include one or more cylindrical rails, e.g., 1105A/1105B, in order to facilitate vertical movement of the mounting device 1100. As discussed herein, smartphone models may have different designs and/or layouts resulting in varying placement of the camera lens 205 thereon. Accordingly, the vertical movement of the mounting device 1100 enables alignment of the camera lens 205 with the lens assembly 105 for a variety of smartphone models while the smartphone is mounted to the mounting assembly 115.

In some embodiments, the mounting apparatus comprises fixation knobs 1110 and/or 1120 to fix or lock the position of components of the imaging adapter 100 to reduce movement after a configuration is set. Knob 1110 may be adjusted to fix the horizontal or lateral position of the mounting plate 1130 by clamping onto rails 1160A/1160B rails at the set position. Likewise, knob 1120 may be adjusted to fix the vertical position of the mounting device 1100 on the mounting assembly 115 by clamping onto rails 1105A/1105B.

The features of the imaging adapter 100 as discussed herein provides a high degree of versatility and efficiency for cooperating with various smartphones for enhanced smartphone imaging. While retinal imaging is particularly discussed herein, it should be understood that the imaging adapter 100 is useful for various types of imaging and particularly suited for applications requiring detailed and precise imaging, e.g., medical examinations, scientific research, and professional photography.

As discussed herein, a notable feature of the optical housing unit 1140 and the cooperating mounting plate 1130 is the 200-degree open area, which provides ample flexibility to adjust the relative orientation of the optical housing unit 1140 and the mounting plate 1130 for alignment. While this feature is useful for aligning the camera lens 205 with the lens assembly 105, it is particularly useful for precisely positioning the flexible waveguide 1145 with respect to a flash unit 210 on the smartphone 201.

The flexible waveguide 1145 is mounted to a holder that is configured to traverse along a rail-slot or slot channel 1150, thereby allowing the flexible waveguide 1145 to be adjusted along a single axis. This movement enables alignment of the receiving end of the flexible waveguide 1145 with the flash unit 210.

The slot channel 1150 may be designed and configured to facilitate free movement of the flexible waveguide 1145. The slot channel 1150 ensures that the waveguide can be accurately aligned with the orientation of the flash unit 210. Accordingly, the design of the slot channel 1150 provides a significant advantage in the adaptability of the imaging adapter 100 to various smartphone models.

In some embodiments, the lens assembly 105 includes a flexible waveguide adjustment knob 1300. The flexible waveguide adjustment knob 1300 enables precise manipulation of the flexible waveguide 1145 to adjust the position of the receiving end along a first axis, thereby allowing greater alignment with the flash unit 210 to efficiently collect high quality images. The flexible waveguide adjustment knob 1300 provides a significant advantage in the adaptability of the imaging adapter 100 to various smartphone models with a range of designs and orientations of the camera lens 205 and the flash unit 210.

In some embodiments, a slider 1310 accompanies the waveguide adjustment knob 1300 to facilitate vertical motion of the flexible waveguide 1145, thereby providing adjustability along an additional axis. The slider 1310 ensures greater alignment with the flash unit 210 to enhance the quality of captured images.

The lens assembly 105 may comprise a rim attachment 1305 to securely connect the lens assembly 105 to the mounting plate 1130 and/or the mounting assembly 115. Secure connection at this junction may be crucial for maintaining the structural integrity of the imaging adapter 100 and ensuring stable operation of the imaging device 200 during collection of images.

The imaging adapter 100 may further comprise a camera hole 1315. This camera hole 1315 may be an aperture strategically positioned to align with the camera lens 205 of the smartphone 201 in order to enable optical imaging using the smartphone 201. The camera hole 1315 may be configured to ensure that the optical path from the lens assembly 105 to the camera lens 205 of the smartphone 201 is unobstructed, thereby enabling collection of clear and precise retinal images therethrough.

The lens assembly 105 may comprise a flexible eye cup 1400 at an end opposite the rim attachment 1305 for interfacing with the eye 10 in a secure manner. The flexible eye cup 1400 may contact surfaces around the eye 10 to stabilize the lens assembly 105 thereon in a manner that is relatively comfortable for the subject, thereby ensuring that the imaging device 200 can remain properly positioned during use.

The lens assembly 105 may include lenses 1405 and 1410 as shown, which may provide an acceptable field of view and clarity for capturing images. The combination of the lenses 1405 and 1410 allows for versatile imaging capabilities. However, it should be understood that additional lenses may be added to the arrangement and/or the lenses may be re-arranged in order to enhance the imaging quality and/or adapt the imaging device 200 to different scenarios with particular imaging requirements.

The lens assembly 105 may further comprise a mirror 1415, which may be positioned at a 45-degree angle with respect to an exit trajectory of the flexible waveguide 1425. The mirror 1415 effectively reflects the light from the flexible waveguide 1425 onto a beam splitter 1420 to accurately direct light through the lens assembly 105 to the imaging site.

The beam splitter 1420 may also be positioned at a 45-degree angle. The beam splitter 1420 may serve a dual purpose. First, it may direct the light received from the flash unit 210 through the flexible waveguide 1425 and mirror 1415 to the eye 10 in order to illuminate the imaging site as discussed herein. Second, light reflected from the imaging site (e.g., from the retina of the eye 10) may travel back towards the beam splitter 1420 and pass therethrough without being deflected. As shown in FIG. 15, the light would be received by the camera lens 205 of the smartphone 201 and be captured in the form of an image. Accordingly, the beam splitter 1420 enables the dual transmission and reception of light to enable effective retinal imaging.

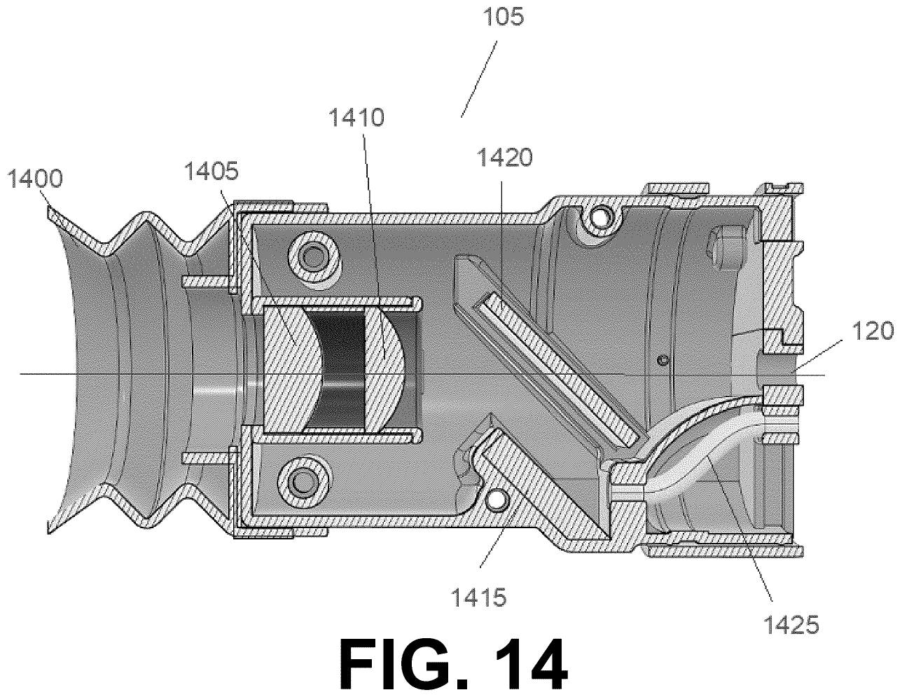

As shown in FIGS. 14-15, the various components of the lens assembly 105 and the manner in which they operate together define a central optical axis 120 along which light is directed from the imaging site (i.e., the eye 10) to the camera lens 205 of the smartphone 201 to precisely capture images.

The flexible waveguide 1425 may be an optical fiber. One end of the flexible waveguide 1425 may be in contact with or adjacent to the flash unit 210, while the opposing end directs light therethrough towards the mirror 1415. This arrangement transfers the light from the flash unit 210 to the mirror 1415, thereby facilitating the illumination necessary for high quality retinal imaging.

The distal end of the lens assembly 105 may comprise component 150, which houses one or more condensing lenses 155. The condensing lenses 155 may function to focus and direct light onto the retina.

The beam splitter 1420 and the mirror 1415 may operate in tandem within the optical path of the lens assembly 105. The mirror 1415, positioned to receive light from the flexible waveguide 1425, reflects this light onto the beam splitter 1420. The beam splitter 1420 then directs this light through the condensing lenses 155 onto the retina 10. Accordingly, light from the flash unit 210 travels through the flexible waveguide 1425 to the mirror 1415 and continues on to the beam splitter 1420. The light then passes through the condensing lenses 155 to illuminate the retina. The reflected light from the retina travels back through these components-passing through the condensing lenses 155 and the beam splitter 1420 and continuing along the central optical axis 120 to ultimately reach the camera lens 205 of the smartphone 201. The pathways created by this specific arrangement of optical elements enables the capture of high-quality retinal images via the smartphone camera. As described, the flexible waveguide 1425 (acting an optical fiber) plays a pivotal role in this process by efficiently transferring light from the flash unit 210 to the eye 10 to ensure the necessary illumination for retinal imaging.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Instead, this application is intended to cover any variations, uses, or adaptations of the present teachings and use its general principles. Further, this application is intended to cover such departures from the present disclosure as come within known or customary practice in the art to which these teachings pertain. Many modifications and variations can be made to the particular embodiments described without departing from the spirit and scope of the present disclosure as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. An apparatus for capturing an image of an eye of a patient, the apparatus comprising:
a lens assembly comprising a first end, a second end, and a central optical axis extending between the first end and the second end, wherein the first end is configured to be aligned with a camera lens of a smart device, and wherein the second end is configured to interface with a surface adjacent the eye of the patient; and
a mounting assembly configured to couple the smart device to the lens assembly to form an imaging device, wherein the mounting assembly is configured to adjust a position of the smart device with respect to the lens assembly,
wherein the lens assembly is configured to transmit light from the eye of the patient to the camera lens when the smart device is coupled to the lens assembly such that the second end is aligned with the camera lens.

2. The apparatus of claim 1, wherein the smart device is one of a smartphone and a tablet.

3. The apparatus of claim 1, wherein the imaging device formed by the apparatus and the smart device is a digital fundus camera.

4. The apparatus of claim 1, wherein an optical axis of the camera lens coincides with the central optical axis when the smart device is coupled to the lens assembly.

5. The apparatus of claim 1, wherein the lens assembly comprises one or more lenses and one or more reflectors.

6. The apparatus of claim 5, wherein the lens assembly further comprises a light channel having a first end and a second end, wherein the first end of the light channel is configured to receive illuminating light from a flash unit of the smart device when the smart device is coupled to the lens assembly, and wherein the second end of the fiber optic tube is configured to emit the illuminating light to the eye of the patient.

7. The apparatus of claim 6, wherein the light channel comprises a fiber optic tube.

8. The apparatus of claim 6, further comprising a beam splitter configured to:
deflect the illuminating light from the light channel to the eye of the patient; and
transmit the light from the eye of the patient to the camera lens.

9. The apparatus of claim 5, further comprises one or more condensing lenses.

10. A system for processing an image of an eye of a patient, the system comprising:
an imaging device comprising a camera lens;
an imaging adapter comprising a lens assembly in optical communication with the camera lens, the lens assembly having a first end, a second end, and a central optical axis extending between the first end and the second end, wherein the first end is configured to be aligned with the camera lens, and wherein the second end is configured to interface with a surface adjacent the eye of the patient;
a processor in electrical communication with the imaging device; and
a non-transitory, computer-readable medium storing instructions that, when executed, cause the processor to:
receive an image of the eye of the patient from the imaging device,
identify one or more features of a retina of the eye using a machine learning algorithm based on the image, and
determine a condition of the patient based on the one or more identified features.

11. The system of claim 10, further comprising a database in electrical communication with the processor, the database comprising ophthalmological data,
wherein the machine learning algorithm identifies one or more features of the retina based further on the ophthalmological data.

12. The system of claim 11, wherein the machine learning algorithm determines the condition of the patient based further on the ophthalmological data.
